(19) United States
(12) Patent Application Publication
(10) Pub. No.: US 2017/0351330 A1
(43) Pub. Date: Dec. 7, 2017

(54) Communicating Information Via A Computer-Implemented Agent

(71) Applicants: John C. Gordon, Newcastle, WA (US); Khuram Shahid, Seattle, WA (US)

(72) Inventors: John C. Gordon, Newcastle, WA (US); Khuram Shahid, Seattle, WA (US)

(21) Appl. No.: 15/174,587

(22) Filed: Jun. 6, 2016

(51) Int. Cl. G06F 3/01; G06F 3/16; A61B 5/04; A61B 5/16; A61B 5/0476; G06K 9/00; A61B 5/00

(52) U.S. Cl. CPC G06F 3/015; G06K 9/00302; G06F 3/167; G06F 3/017; A61B 5/0015; A61B 5/165; A61B 5/0476; A61B 5/04012; A61B 5/486

(57) ABSTRACT

Techniques and systems for communicating information via a computer-implemented agent are described. A computing device may obtain sensor data of an individual, such as visual data, audible data, physiological data, or combinations thereof. An emotional state of the individual may be determined based on the sensor data. A communications framework may be identified based on the emotional state of the individual. The communications framework may indicate a manner in which the computer-implemented agent communicates information to the individual. For example, the communications framework may specify voice features, facial features, body language, positioning in the environment, or combinations thereof, that may be utilized to produce a representation of a computer-implemented agent that communicates information to the individual. In some cases, the individual may provide feedback indicating a preference to have the computer-implemented agent communicate information in a different manner.

### BACKGROUND

Computing devices are often utilized to communicate information to individuals. In some cases, the individual may request specific information via a computing device. For example, an individual may enter search terms in a browser application for a search engine to obtain information related to the search terms. The individual may then navigate to webpages provided by the search engine using the browser application. In other cases, the computing device may be set to automatically provide information to an individual. To illustrate, a computing device may provide alarms or notifications to an individual. In certain situations, a computing device may utilize a voice activated agent to obtain information on behalf of the individual. In an example, an individual may ask the computer-implemented agent to obtain information related to particular keywords. As a result, the computer-implemented agent may provide visual and/or audible information to the individual using output devices of the computing device.

### SUMMARY

Techniques and systems for communicating information via a computer-implemented agent are described. In particular, a computing device may obtain sensor data of an individual, such as visual data, audible data, physiological data, or combinations thereof. An emotional state of the individual may be determined based on the sensor data. A communication framework may be identified based on the emotional state of the individual. The communication framework may indicate a manner in which the computer-implemented agent communicates information to the individual. For example, the communication framework may specify voice features, facial features, body language, positioning in the environment, or combinations thereof, that may be utilized to produce a representation of a computer-implemented agent that communicates information to the individual. In some scenarios, the individual may provide feedback indicating a preference to have the computer-implemented agent communicate information in a different manner.

This Summary is provided to introduce a selection of concepts in a simplified form that are further described below in the Detailed Description. This Summary is not intended to identify key or essential features of the claimed subject matter, nor is it intended to be used to limit the scope of the claimed subject matter.

### BRIEF DESCRIPTION OF THE DRAWINGS

The Detailed Description is set forth with reference to the accompanying figures, in which the left-most digit of a reference number identifies the figure in which the reference number first appears. The use of the same reference numbers in the same or different figures indicates similar or identical items or features.

FIG. 1 is a diagram of an example environment to communicate information via a computer-implemented agent.

FIG. 2 is a diagram indicating different communication frameworks for a computer-implemented agent to communicate information based on an emotional state of an individual.

FIG. 3 is a diagram illustrating an example environment to obtain feedback from an individual to modify a communication framework used by a computer-implemented agent to communicate information.

FIG. 4 is a block diagram illustrating an example system to communicate information via a computer-implemented agent.

FIG. 5 is a flowchart of a first example process to communicate information via a computer-implemented agent.

FIG. 6 is a flowchart of a second example process to communicate information via a computer-implemented agent.

FIG. 7 is a schematic diagram illustrating an example computer architecture usable to implement aspects of communicating information via a computer-implemented agent.

FIG. 8 is a schematic diagram illustrating an example distributed computing environment capable of implementing aspects of communicating information via a computer-implemented agent.

FIG. 9 is a schematic diagram illustrating another example computing device architecture usable to implement aspects of communicating information via a computer-implemented agent.

### DETAILED DESCRIPTION

Described herein are systems and processes to communicate information via a computer-implemented agent. In particular, the computer-implemented agent may communicate information to an individual based at least partly on an emotional state of the individual. Data obtained from one or more sensors may be utilized to determine an emotional state of an individual. In an example, one or more cameras may capture images of an individual and determine an emotional state of the individual based at least partly on the images. To illustrate, facial expressions and/or gestures of the individual may be analyzed to determine an emotional state of an individual. Additionally, one or more microphones may capture audible data from the individual. The audible data of the individual may be analyzed to identify one or more words, sounds, voice characteristics (e.g., tone, pitch, volume), or combinations thereof to determine an emotional state of an individual. Further, physiological data of the individual may be analyzed to determine an emotional state of the individual. In some implementations, electroencephalography (EEG) data may be analyzed to determine an emotional state of the individual. In addition, heart-related characteristics, body temperature, skin characteristics, breathing characteristics, muscle activity, combinations thereof, and the like, may be analyzed to determine an emotional state of the individual.

The emotional state of an individual may be utilized to determine a communication framework by which a computer-implemented agent may communicate information to the individual. The communication framework may indicate features of a computer-implemented agent that may be used to communicate information to the individual. In some cases, the features of the computer-implemented agent during communication of information may include voice features. The voice features may include tone, pitch, volume, and pace. The features of the agent during communication of information may also include facial features. The facial features may include mouth features (e.g., smiling, frowning, open, closed), nose features (e.g., crinkled nose, twitching nose), eye features (e.g., closed eyes, wink, wide open eyes, raised eyebrow(s), squint), other facial features (e.g., furrowed brow), combinations thereof, and so forth.

Additionally, the features of the computer-implemented agent during the communication of information may include body language. Body language may include gestures (e.g., pointing, “follow me” gesture), arm positioning (e.g., hand(s) on head, hand on chin (thinking pose), hands on hips), leg positioning (e.g., one leg in front of the other, standing, sitting), head positioning (e.g., tilted to one side, bowed), shoulder positioning (e.g., slumped, straight up), combinations thereof, and the like. In some cases, the body language features may be combined to produce a pose, such as hands on hips with head tilted to the side. Further, the features of the computer-implemented agent during the communication of information may include positioning of the computer-implemented agent within an environment. For example, the computer-implemented agent may be positioned in close proximity to the individual, such as within an arm's length. In another example, the computer-implemented agent may be positioned several feet away from the individual.

After determining an emotional state of an individual and a communication framework that corresponds to the emotional state, a representation of the computer-implemented agent may be generated. The representation may communicate information to the individual according to the communication framework. The representation may include one or more 3-dimensional images of the computer-implemented agent that express visible characteristics, audible characteristics, or both corresponding to the features of the communication framework. In other cases, the representation may include one or more 2-dimensional images of the computer-implemented agent that express visible characteristics, audible characteristics, or both corresponding to the features of the communications framework. The representation may be displayed on a display device accessible to the individual. In various implementations, the display device may be associated with a computing device, such as a mobile phone, a laptop computing device, a tablet computing device, a gaming console, a desktop computing device, a wearable computing device (e.g., head-mounted display, glasses, watch, fitness tracking device, etc.), combinations thereof, and the like. In particular, implementations, the representation may be projected into an environment. Additionally, audible communications may also be associated with the representation to communicate information to the individual.

In some instances, the communication framework may be modified based on preferences of an individual. For example, an individual may provide feedback regarding the manner in which a computer-implemented agent communicated information to the individual. In particular implementations, the feedback may be expressly provided by the individual. To illustrate, the individual may provide words and/or gestures to indicate feedback regarding the manner in which the computer-implemented agent communicated information to the individual. In an illustrative example, the individual may indicate that a voice of the computer-implemented agent is too loud or that the voice of the computer-implemented agent is too harsh. In another illustrative example, the individual may indicate that the representation of the computer implemented agent is displayed too close to the individual. In various implementations, the computer-implemented agent may request feedback from the individual regarding the manner in which information was communicated to the individual. In other implementations, the individual may provide indirect feedback that is used to infer preferences of the individual. In some illustrative examples, the individual may have a furrowed brow or a surprised expression that may be used to infer that the manner in which the computer-implemented agent communicated information was not preferred by the individual.

In an illustrative implementation, sensor data may be analyzed to determine that an emotional state of an individual is characterized as happy. In this situation, the computer-implemented agent may communicate information at a somewhat loud volume with an upbeat tone, and at a relatively fast pace. In addition, the computer-implemented agent may have a smiling facial expression and have animated body movements. In another illustrative implementation, sensor data may be analyzed to determine that an emotional state of an individual is characterized as sad. In this scenario, the computer-implemented agent may communicate information at a relatively lower volume and a relatively slower pace with a softer tone. Further, the computer-implemented agent may have non-expressive or soft facial features and have few body movements.

By utilizing physiological data to determine an emotional state of an individual, the processes and systems described herein provide a more accurate determination of the emotional state of the individual than typical systems and processes. In particular, EEG data has been obtained by scientists showing activity in areas of the brain that correspond with certain emotional states. Additionally, obtaining feedback from the individual regarding the manner in which the computer-implemented agent communicates information to the individual may improve the effectiveness of the communication of information to the individual by the computer-implemented agent because the interactions between the individual and the computer-implemented agent may be customized. Further, by determining an emotional state of an individual before causing a computer-implemented agent to communicate with the individual may help the computer-implemented agent to provide communications that are considered empathetic by the individual. Also, in some implementations, at least a portion of the operations performed to determine the emotional state of the individual may be performed by a computing device that is located remote from a computing device of the individual. In this way, the amount of computing resources and/or memory resources of the computing device of the individual may be minimized. Thus, the form factor of the computing device of the individual may be smaller and more lightweight than a computing device that includes an increased number of computing resources and/or memory resources.

These and various other example features will be apparent from a reading of the following description and a review of the associated drawings. However, the claimed subject matter is not limited to implementations that solve any or all disadvantages or provide any of the benefits noted in any part of this disclosure.

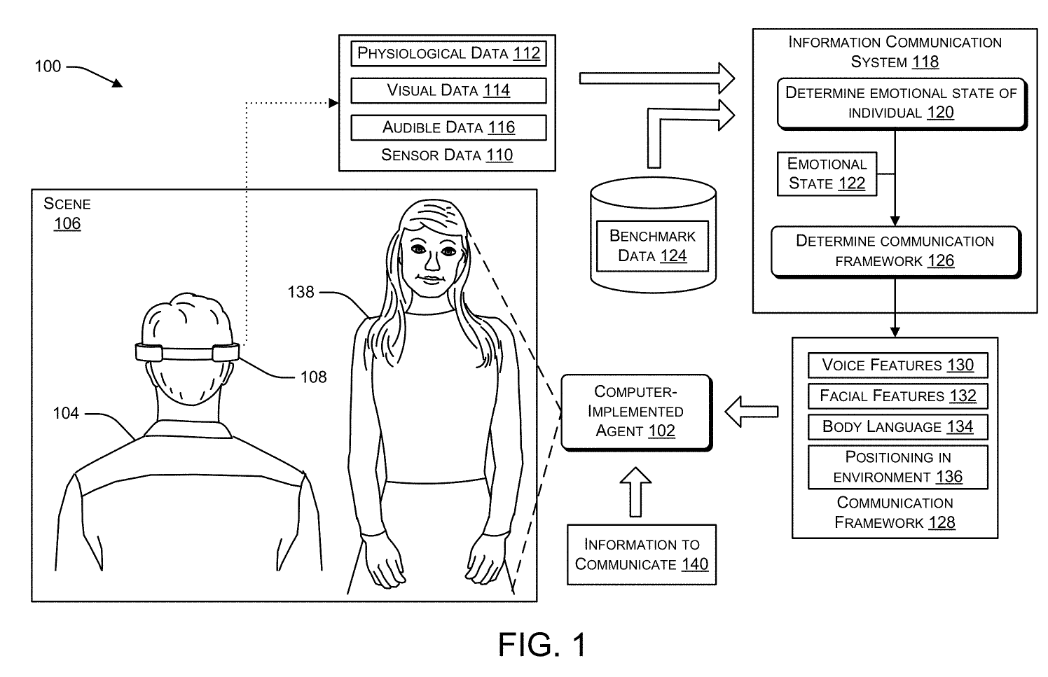

FIG. 1 is a diagram of an example environment 100 to communicate information via a computer-implemented agent 102. The computer-implemented agent 102 may include software, hardware, firmware, or combinations thereof, that are utilized to perform actions on behalf of an individual 104 positioned in a scene 106. For example, the computer-implemented agent 102 may obtain information on behalf of the individual 104, such as performing a search for the individual 104 according to certain criteria provided by the individual 104. In another example, the computer-implemented agent 102 may cause computing devices to perform one or more operations. To illustrate, the computer-implemented agent 102 may cause an electronic thermostat to modify the temperature in a residence of the individual 104. In another illustration, the computer-implemented agent 102 may cause a television to turn to a particular channel or cause a digital recording device to record a particular television program.

The scene 106 may be a real-world scene that includes tangible, physical objects. In other cases, the scene 106 may be a mixed reality scene that includes objects that are tangible, physical objects and that includes computer-generated images of objects. Additionally, the scene 106 may be a virtual reality scene including objects are computer-generated.

The environment 100 also includes a computing device 108. In the illustrative example of FIG. 1, the computing device 108 is a wearable computing device. In some cases, the computing device 108 may include glasses. In other instances, the computing device 108 may include a headset computing device, such as a head mounted display. Although, the computing device 108 is shown in the illustrative example of FIG. 1 as a wearable computing device, in other scenarios, the computing device 108 may include a mobile telephone, a tablet computing device, a laptop computing device, a portable gaming device, a gaming console, a television, or combinations thereof.

The computing device 108 may include one or more sensors to obtain sensor data 110. The sensor data 110 may include physiological data 112, visual data 114, and audible data 116. Although the illustrative example of FIG. 1 shows that the sensor data 110 includes physiological data 112, visual data 114, and audible data 116, in other implementations, the sensor data 110 may include one or more of the physiological data 112, the visual data 114, or the audible data 116. The physiological data 112 may indicate measurements related to physiological processes of the individual 104. In some cases, the physiological data 112 may indicate heart activity of the individual 104, brain activity of the individual 104, lung activity of the individual 104, muscle activity of the individual 104, body temperature of the individual 104, skin characteristics of the individual 104, or combinations thereof. In a particular example, the computing device 104 may include one or more sensors to capture EEG data of the individual 104.

The computing device 108 may also include one or more sensors to obtain visual data 114. The visual data 114 may include one or more images related to the scene 106. For example, the visual data 114 may include one or more images of the individual 104. To illustrate, the visual data 114 may include one or more images of the face of the individual 104, one or more images of at least one eye of the individual 104, one or more images of limbs of the individual 104, one or more images of at least one hand of the individual 104, one or more images of at least one foot of the individual 104, or combinations thereof. The visual data 114 may include one or more images of objects included in the scene 106, one or more images of additional individuals in the scene 106, one or more images of representations of the computer-implemented agent 102, or combinations thereof. In particular implementations, the computing device 108 may include one or more cameras to capture images of the scene 106. In an example, the computing device 108 may include a user facing camera that captures images of the individual 104. In addition, the computing device 108 may include an environment-facing camera that captures images of the scene 106. The computing device 108 may also include one or more depth sensing cameras.

The computing device 108 may include one or more sensors to obtain audible data 116. The audible data 116 may include sounds related to the scene 106. In some cases, the audible data 116 may include sounds produced by the individual 102. The audible data 116 may also include sounds produced by additional individuals in the scene 106. In addition, the audible data 116 may include sounds produced by one or more objects in the scene 106. Further, the audible data 116 may include sounds generated by the computer-implemented agent 102. In particular implementations, the computing device 108 may include one or more microphones to capture sounds in the scene 106.

In some implementations, the sensor data 110 may be provided to an information communication system 118. The information communication system 118 may include software, hardware, firmware, or combinations thereof, to provide information to the individual 104. The information communication system 118 may analyze the sensor data 110 to determine a manner in which to communicate information to the individual 104. In some implementations, at least a portion of the information communication system 118 may be implemented by the computing device 108. In other implementations, at least a portion of the information communication system 120 may be implemented by one or more additional computing devices. The one or more additional computing devices may be located in a location that is remote from the computing device 108. In various implementations, the one or more additional computing devices may be located in a location that is proximate to the computing device 108.

In the illustrative example of FIG. 1, at 120, the information communication system 118 may analyze the sensor data 110 to determine an emotional state 122 of the individual 104. In some implementations, the information communication system 118 may compare the sensor data 110 to benchmark data 124 that may characterize particular emotional states. For example, the information communication system 118 may obtain benchmark data 124 indicating EEG patterns that correspond to different emotional states. To illustrate, the benchmark data 124 may include one or more first EEG patterns that correspond to a first emotional state and one or more EEG patterns that correspond to a second emotional state. In another example, the information communication system 118 may obtain benchmark data 124 including images of facial features that correspond to different emotional states. In an illustrative scenario, the benchmark data 124 may include first images that include a first set of facial features that correspond with a first emotional state and second images that includes a second set of facial features that correspond with a second emotional state. In an additional example, the information communication system 118 may obtain benchmark data 124 related to sounds and/or one or more words that correspond with different emotional states. In another illustrative scenario, the benchmark data 124 may include first sounds having a first set of sound characteristics that correspond with a first emotional state and second sounds having a second set of sound characteristics that correspond with a second emotional state.

In an illustrative implementation, the information communication system 118 may analyze the physiological data 112, the visual data 114, the audible data 116, or combinations thereof, with respect to the benchmark data 124 to determine the emotional state 122. In some cases, the information communication system 118 may determine that the physiological data 112 corresponds with a portion of the benchmark data 124 that includes physiological data associated with the emotional state 122. The information communication system 118 may also determine that the visual data 114 corresponds with a portion of the benchmark data 124 that includes visual data associated with the emotional state 122. In addition, the information communication system 118 may determine that the audible data 116 corresponds with a portion of the benchmark data 124 that includes audible data associated with the emotional state 122.

The information communication system 118, at 126, may utilize the emotional state 122 to determine a communication framework 128 from among a number of communication frameworks. The communication framework 128 may include features of the computer-implemented agent 102 that may be used to communicate information to the individual 104. The communication framework 128 may correspond with the emotional state 122. That is, at least one communication framework of the plurality of communication frameworks may correspond with an emotional state of a plurality of emotional states. In some cases, the communication framework 128 may correspond with the individual 104. That is, the components of the communication framework 128 may be customized according to preferences of the individual 104. In this way, different individuals may be associated with communication frameworks that are utilized by the computer-implemented agent 102 to communicate information in different ways based at least partly on the preferences of a particular individual with whom the computer-implemented agent 102 is communicating.

In some implementations, the communication framework 128 may include voice features 130. The voice features 130 may be related to the manner in which the computer-implemented agent 102 speaks with the individual 104. For example, the voice features 130, such as include tone, pitch, volume, pace, or combinations thereof. In some cases, the voice features 130 may relate to sounds and/or words used by the computer-implemented agent 102 to communicate with the individual 104. The voice features 130 may also relate to audible characteristics of the voice of the computer-implemented agent 102 as words and/or sounds are communicated to the individual 104.

The communication framework 128 may also include facial features 132. The facial features 132 may relate to an appearance of the face of representations of the computer-implemented agent 102. In some implementations, the facial features 132 may relate to an appearance of the eyes of the computer-implemented agent 102, an appearance of a nose of the computer-implemented agent 102, an appearance of a mouth of the computer-implemented agent 102, or combinations thereof. Additionally, the facial features 132 may relate to other portions of the face of the computer-implemented agent 102, such as cheeks, chin, eyebrows, forehead, combinations thereof, and the like.

In addition, the communication framework 128 may include body language 134. The body language 134 may indicate an arrangement of various body parts of the computer-implemented agent 102. The body language 134 may also indicate motion of body parts of representations of the computer-implemented agent 102. In an example, the body language 134 may indicate a position of one or more hands of the computer-implemented agent 102, a position of one or more fingers of the computer-implemented agent 102, a position of one or more arms of the computer-implemented agent 102, a position of one or more legs of the computer-implemented agent 102, a position of one or more feet of the computer-implemented agent 102, a position of one or more shoulders of the computer-implemented agent 102, a posture of the computer-implemented agent 102, other arrangements of a body of the computer-implemented agent 102, or combinations thereof.

Further, the communication framework 128 may include positioning in environment 136. The positioning in environment 136 of the computer-implemented agent 102 may relate to a location of representations of the computer-implemented agent 102 within the scene 106. In some cases, the positioning in environment 136 of the computer-implemented agent 102 may correspond with a proximity of the computer-implemented agent 102 with respect to the individual 104. In an example, the positioning in environment 136 of the computer-implemented agent 102 may indicate a distance from the individual 104 in which the computer-implemented agent 102 is located. In another example, the positioning in environment 136 may relate to the field of view of the individual 104. For example, the positioning in environment 136 may indicate that the computer-implemented agent 102 is to be fully within the field of view of the individual 104, outside of the field of view of the individual 104, just inside the field of view of the individual, 106, or combinations thereof.

The communication framework 128 may be utilized by the computer-implemented agent 102 to generate an example visual representation 138 of the computer-implemented agent 102. The visual representation 138 may include one or more 3-dimensional images of the computer-implemented agent 102 or one or more 2-dimensional images of the computer-implemented agent 102. The visual representation 138 may be projected into the scene 106, in some cases. In other instances, the visual representation 138 may be displayed on a display device. In an illustrative implementation, the visual representation 138 may be displayed on a display device associated with the computing device 108. Furthermore, the computer-implemented agent 102 may generate audible output, such as via one or more speakers, in order to communicate information to the individual 104. The visual representation 138 may indicate facial movement of the computer-implemented agent 102 to correspond with the audible output being provided.

In addition to generating the visual representation 138 in accordance with the communication framework 128, the visual representation 138 may also convey other visual features of the computer-implemented agent 102, such as a size of the computer-implemented agent 102 (e.g., height, weight), a gender of the computer-implemented agent 102, hair style and hair color of the computer-implemented agent 102, skin tone of the computer-implemented agent 102, combinations thereof, and so forth.

The computer-implemented agent 102 may also obtain information to communication 140 to the individual 104. The information to communicate 140 may be obtained from one or more computing devices. In some cases, the information to communicate 140 may relate to information obtained by the computer-implemented agent 102 on behalf of the individual 104. For example, information to communicate 140 may include search results obtained by the computer-implemented agent 102 on behalf of the individual 104 based at least partly on one or more search criteria. In other cases, the information to communicate 140 may be provided by an additional computing device in association with an application executing on the additional computing device. To illustrate, the information to communicate 140 may include directions to a destination provided by a geographic positioning system (GPS) executed by a mobile phone of the individual 104. The information to communicate 140 may also include notifications for the individual 104. In a particular example, the information to communicate 140 may include a notification that a message, such as an email, Short Message Service (SMS) message, or a Multimedia Messaging Service (MMS) message, has been received that is associated with the individual 104. In other examples, the information to communicate 140 may include reminders of events, alarms, other notifications, or combinations thereof.

In some implementation, the communication framework 128 and/or the representation 138 of the computer-implemented agent 102 may be based at least partly on the information to communicate 140. In an example, the information to communicate 140 may include a warning for the individual 104 to avoid danger. In this scenario, the communication framework 128 may take into account the emotional state 122 of the individual 104 and also the nature of the information to communicate 140. Thus, the representation 138 of the computer-implemented agent 102 may communicate the information to communicate 140 in a manner that will get the attention of the individual 104, such as using a loud, high-pitched voice and dramatic gestures.

Although not shown in the illustrative example of FIG. 1, in particular implementations, the communication framework 128 and/or the representation 138 of the computer-implemented agent 102 may be based at least partly on an activity being performed by the individual 104. For example, the information communication system 118 may determine that a particular communication framework is to be utilized based at least partly on determining that the individual 104 is engaged in a particular activity. To illustrate, the information communication system 118 may analyze one or more of the physiological data 112, the visual data 114, or the audible data 116 to determine the individual 104 is engaged in a particular activity and identify a communication framework corresponding to the particular activity. The computer-implemented agent 102 may then generate the representation 138 based at least partly on the communication framework corresponding to the particular activity. In an illustrative example, the information communication system 118 may analyze the sensor data 110 and determine that the individual 104 is engaged in an exercise activity. Continuing with this example, the information communication system 118 may identify a communication framework that is to be provided to the individual 104 during a period of time that the individual 104 is exercising. In another illustrative example, the information communication system 118 may analyze the sensor data 110 and also monitor one or more applications being utilized by the individual 104. In particular, the information communication system 118 may determine that the individual 104 is listening to music via a media player application and identify a communication framework to utilize to generate the representation 138 of the computer-implemented agent 102 in response to determining that the individual 104 is listening to music.

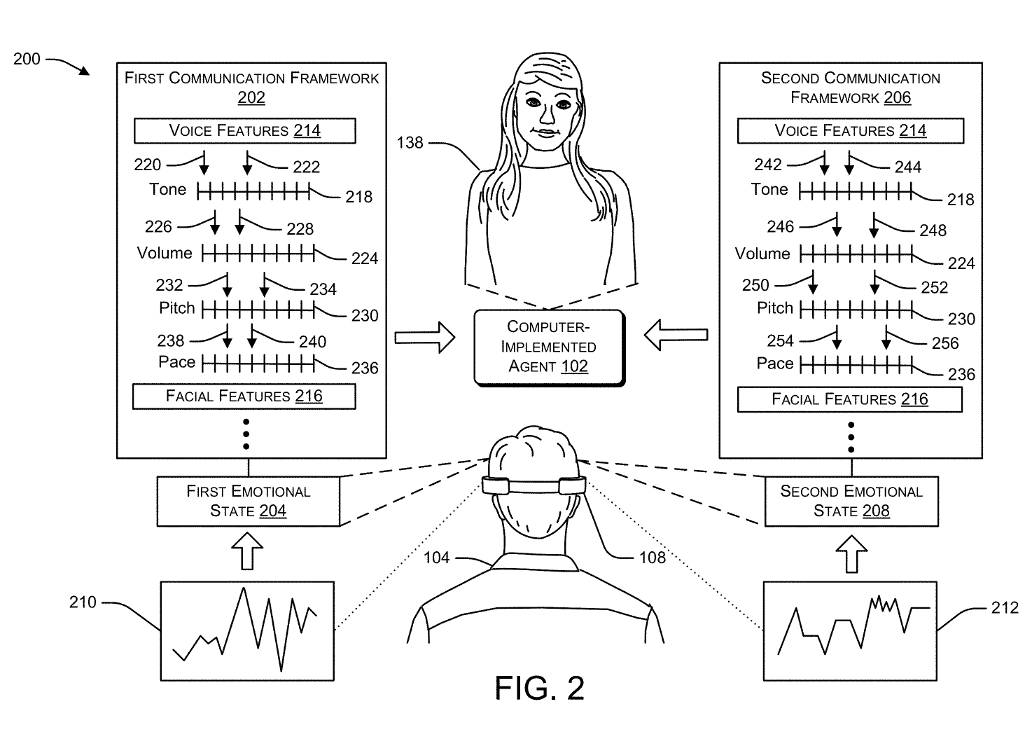

FIG. 2 is a diagram indicating different communication frameworks for a computer-implemented agent 102 to communicate information based on an emotional state of an individual 104. The computer-implemented agent 102 and the individual 104 may be located in an environment 200. In the illustrative example of FIG. 2, the representation 138 of the computer-implemented agent 102 may be based at least partly on a first communication framework 202 corresponding to a first emotional state 204 or a second communication framework 206 corresponding to a second emotional state 208. The first emotional state 204 may be associated with first sensor data obtained by the computing device 108, such as a first EEG pattern 210. The second emotional state 208 may be associated with second sensor data obtained by the computing device 108, such as a second EEG pattern 212. The first EEG pattern 210 and the second EEG pattern 212 may indicate brain activity of the individual 104 over a period of time. In some implementations, the first EEG pattern 210 and the second EEG pattern 212 may represent voltages measured by one or more sensors of the computing device 108. Although the illustrative implementation of FIG. 2 shows that the first emotional state 204 is related to the first EEG pattern 210 and that the second emotional state 208 is related to the second EEG pattern 212, the first emotional state 204 and the second emotional state 208 may also be related to other sensor data, such as visual sensor data and/or audible sensor data.

The first communication framework 202 and the second communication framework 206 may include one or more components that may be used to determine physical features of the computer-implemented agent 102 that are expressed by the representation 138. In the illustrative example of FIG. 2, the first communication framework 202 and the second communication framework 206 may include at least voice features 214 and facial features 216. The first communication framework 202 and the second communication framework 206 may also include other components, such as body language features and/or positioning in environment features. Each of the components of the first communication framework 202 and the second communication framework 206 may include one or more subcomponents that correspond to attributes of the components that may be adjusted to generate the physical appearance of the representation 138 and/or to generate sound provided by the computer-implemented agent 102.

In some cases, the subcomponents of each component of the first communication framework 202 and the second communication framework 206 may be quantified to indicate different states for each subcomponent. For example, each of the subcomponents may be associated with a scale, a lower threshold, and an upper threshold. The scale may indicate a range of values corresponding to a continuum of states for a respective subcomponent. The states of some subcomponents may represent a set of visible features of an aspect of the appearance of the representation 138. For example, the states of a subcomponent related to the mouth of the computer-implemented agent 102 may indicate different configurations of the mouth of the representation 138 of the computer-implemented agent 102. In another example, the states of a subcomponent related to eyes of the representation 138 may indicate different positions of the pupils and irises of the eyes of the representation 138 and/or positions of lids of the eyes of the representation 138. In addition, the states of some components may indicate an aspect of a location of the representation 138 in the environment 200. To illustrate, the states of a subcomponent related to proximity to the individual 104 may indicate distances from the individual 104.

The voice features 214 of the first communication framework 202 and the second communication framework 206 may correspond to audible characteristics of communications produced by the computer-implemented agent 102. In the illustrative example of FIG. 2, the voice features 214 may be associated with the subcomponents of tone, volume, pitch, and pace. The subcomponent of tone may be associated with a first scale 218, a first lower threshold 220, and a first upper threshold 222. As the values move along the first scale 218 from left to right, which represents least to greatest values, the tone may change from being considered a soft tone to a harsher tone. In some cases, the tone may correspond with words, sounds, a sharpness of voice, or a combination thereof, used by the computer-implemented agent 102 to communicate with the individual 104.

The subcomponent of volume may be associated with a second scale 224, a second lower threshold 226, and a second upper threshold 228. As the values move along the second scale 224 from left to right, which represents least to greatest values, the volume may change from a low volume to a higher volume. In some cases, the volume may correspond to a number of decibels measured for one or more sounds with an increasing volume corresponding to an increasing number of decibels. In addition, the subcomponent of pitch may be associated with a third scale 230, a third lower threshold 232, and a third upper threshold 234. As the values move along the third scale 230 from left to right, which represents lowest to highest notes on a music scale, the pitch may change from corresponding to lower notes to higher notes. Further, the subcomponent of pace may be associated with a fourth scale 236, a fourth lower threshold 238, and a fourth upper threshold 240. As the values move along the fourth scale 236 from left to right, which represents least to greatest rates, the pace may change from a relatively slow pace to a relatively fast pace. In some implementations, the pace may correspond to a rate at which sounds are produced by the representation 138 of the computer-implemented agent 102 within a specified period of time and/or the number of words produced by the representation 138 of the computer-implemented agent 102 within a specified period of time.

The second communication framework 206 may include one or more components of the first communication framework 202. Additionally, the second communication framework 206 may include one or more of the subcomponents of the first communication framework 202. In the illustrative example of FIG. 2, the second communication framework 206 and the first communication framework 202 both include at least components associated with the voice features 214 and the facial features 216 with the voice features 214 including the subcomponents of tone, volume, pitch, and pace. The second communication framework 206 also includes the first scale 218, the second scale 224, the third scale 230, and the fourth scale 236. The values of the subcomponents of the voice features 214 for the second communication framework 206 differ from those of the first communication framework 202. For example, the lower threshold and upper threshold for tone are different for the first communication framework 202 and the second communication framework 206. To illustrate, the tone subcomponent of the second communication framework 206 may have an additional first lower threshold 242 that has a greater value than the first lower threshold 220 and an additional first upper threshold 244 that has a greater value than the first upper threshold 222. Also, the volume subcomponent of the second communication framework 206 may have an additional second lower threshold 246 that has a greater value than the second lower threshold 226 and an additional second upper threshold 248 that has a greater value than the second upper threshold 228. In addition, the pitch subcomponent of the second communication framework 206 may have an additional third lower threshold 250 that has a lower value than the third lower threshold 232 and an additional third upper threshold 252 that has a greater value than the third upper threshold 234. Further, the pace subcomponent of the second communication framework 206 may have an additional fourth lower threshold 254 that has a greater value than the fourth lower threshold 238 and an additional fourth upper threshold 256 that has a greater value than the fourth upper threshold 240. In this way, the voice features 214 used by the computer-implemented agent 102 to communicate information to the individual 104 may be different when the individual 104 is associated with the first emotional state 204 and the second emotional state 208.

By having different communication frameworks associated with different emotional states, the computer-implemented agent 102 may communicate with the individual 104 in a manner that corresponds with a particular emotional state of the individual 104 at a given time. Thus, the appearance of the representation 138 may change as the emotional state of the individual 104 changes according to various communication frameworks. Additionally, the audible characteristics of the computer-implemented agent 102 may be modified as the emotional state of the individual 104 changes.

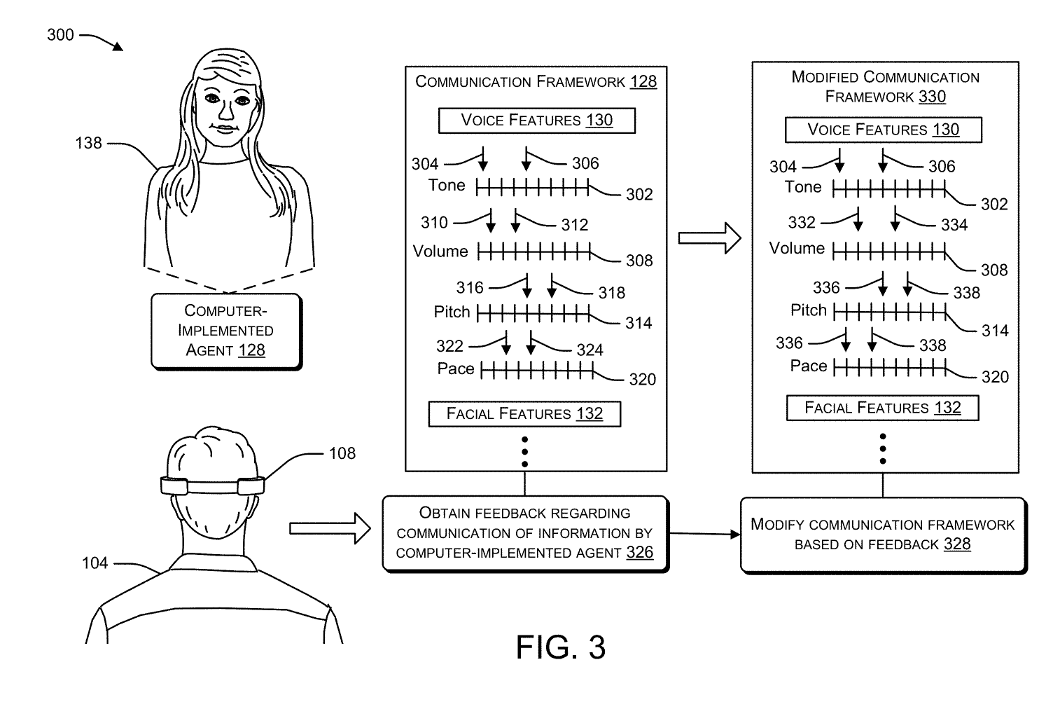

FIG. 3 is a diagram illustrating an example environment 300 to obtain feedback from an individual 104 to modify a communication framework 128 used by a computer-implemented agent 102 to communicate information to the individual 104. The communication framework 128 may include one or more components that may be used to determine physical features of the computer-implemented agent 102 that are expressed by the representation 138. In the illustrative example of FIG. 3, the communication framework 128 may include at least voice features 130 and facial features 132. The communication framework 128 may also include other components, such as body language, positioning in environment, and the like. Each of the components of the communication framework 128 may include one or more subcomponents that correspond to attributes of the components that may be adjusted to generate the physical appearance of the representation 138 and/or to generate sound provided by the computer-implemented agent 102.

In some cases, the subcomponents of each component of the communication framework 128 may be quantified to indicate different states for each subcomponent. For example, each of the subcomponents may be associated with a scale, a lower threshold, and an upper threshold. The scale may indicate a range of values corresponding to a continuum of states for a respective subcomponent. The states of some subcomponents may represent a set of visible features of an aspect of the appearance of the representation 138. In addition, the states of some subcomponents may indicate an aspect of a location of the representation 138 in the environment 300.

The voice features 130 of the communication framework 128 may correspond to audible characteristics of communications produced by the computer-implemented agent 102. In the illustrative example of FIG. 3, the voice features 130 may be associated with the subcomponents of tone, volume, pitch, and pace. The subcomponent of tone may be associated with a first scale 302, a first lower threshold 304, and a first upper threshold 306. The subcomponent of volume may be associated with a second scale 308, a second lower threshold 310, and a second upper threshold 312. In addition, the subcomponent of pitch may be associated with a third scale 314, a third lower threshold 316, and a third upper threshold 318. Further, the subcomponent of pace may be associated with a fourth scale 320, a fourth lower threshold 322, and a fourth upper threshold 324.

At 326, a computing device, such as the computing device 108 may obtain feedback regarding the communication of information by the computer-implemented agent 102. In some cases, the feedback may be obtained from the individual 104. Additionally, the feedback may be obtained by one or more input devices of the computing device 108. In particular implementations, the feedback may include audible feedback. The audible feedback may include one or more sounds, one or more words, or a combination thereof. The feedback may also include visual feedback. The visual feedback may include facial expressions, gestures, body movements, or combinations thereof. In various implementations, the feedback may be electronic feedback. The electronic feedback may be obtained via one or more applications of the computing device 108, one or more user interfaces provided by the computing device 108, or combinations thereof.

In an illustrative example, the feedback may indicate that the individual 104 was dissatisfied with the manner in which the computer-implemented agent 102 communicated information to the individual 104. For example, the feedback may indicate that the computer-implemented agent 102 communicated information to the individual 104 with a volume that is too loud. In another example, the feedback may indicate that the computer-implemented agent 102 communicated information to the individual 104 at a pace that was too fast. In an additional example, the feedback may indicate that the computer-implemented agent 102 is positioned too close to the individual 108.

Based at least partly on the feedback obtained about the manner in which the computer-implemented agent 102 communicated with the individual 104, at 328, the communication framework 128 may be modified to produce a modified communication framework 330. The modified communication framework 330 may include at least some of the components of the communication framework 128. To illustrate, the modified communication framework 330 may include at least the voice features 130 and the facial features 132. Additionally, the modified communication framework 330 may include at least some of the subcomponents of the components of the communication framework 128. In the illustrative example of FIG. 3, the modified communication framework 330 includes the subcomponents of tone, volume, pitch, and pace for the voice features 130. The modified communication framework 330 also includes the first scale 302, the second scale 308, the third scale 314, and the fourth scale 320. The values of one or more of the subcomponents of the voice features 130 for the modified communication framework 330 may differ from those of the communication framework 128. For example, the lower and upper thresholds for volume are different for the communication framework 128 and the modified communication framework 330. To illustrate, the volume subcomponent of the modified communication framework 330 may have an additional second lower threshold 332 that has a greater value than the second lower threshold 310 and an additional second upper threshold 334 that has a greater value than the second upper threshold 312. Also, the pace subcomponent of the modified communication framework 330 may have an additional fourth lower threshold 336 that has a lower value than the fourth lower threshold 322 and an additional fourth upper threshold 338 that has a lower value than the fourth upper threshold 324. The values for the lower threshold and the upper threshold for the tone subcomponent and the pitch subcomponent remain the same in the modified communication framework 330 as the communication framework 128.

Furthermore, the computer-implemented agent 102 may request specific feedback from the individual 104. For example, the computer-implemented agent 102 may ask the individual 104 whether information communicated to the individual 104 was provided in a manner that was unsatisfactory to the individual 104. In some cases, the computer-implemented agent 102 may obtain express feedback from the individual 104 regarding how to modify the behavior of the computer-implemented agent 102 to correspond with preferences of the individual 104. In an illustrative example, the computer-implemented agent 102 may ask whether the individual 104 did not understand information communicated to the individual 104 by the computer-implemented agent 102. In another illustrative example, the computer-implemented agent 102 may ask whether the individual 104 was unable to understand the meaning of the words used to communicate information to the individual 104. In particular implementations, a communication framework may be modified based on the response provided by the individual 104 to the questions provided by the computer-implemented agent 102. To illustrate, the volume of speech may be increased for a communication framework associated with an emotional state where the individual 104 provided express feedback that the individual 104 was unable to hear the words produced by the computer-implemented agent 102. In various implementations, the computer-implemented agent 102 may also provide an apology to the individual 104 in response to determining that the individual 104 is not satisfied with an interaction with the computer-implemented agent 102.

By modifying communication frameworks based on feedback received from an individual, the manner in which information is communicated to the individual by a computer-implemented agent may be customized. Thus, each individual communicating with a computer-implemented agent may be associated with one or more communication frameworks that are different from communication frameworks of one or more other individuals communicating with the computer-implemented agent. In this way, the experience of individuals with the computer-implemented agent may be improved as feedback regarding interactions between the computer-implemented agent and individuals is obtained.

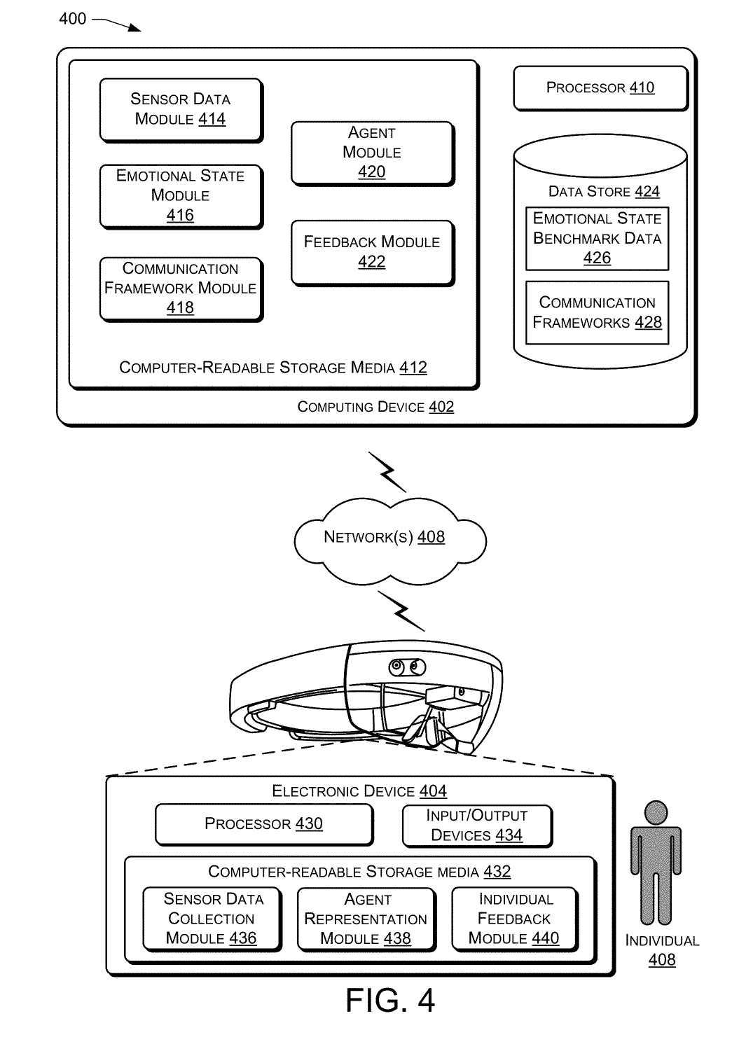

FIG. 4 is a block diagram illustrating an example system 400 to communicate information via a computer-implemented agent. The system 400 includes a computing device 402 that may be used to perform at least a portion of the operations to communicate information to individuals using a computer-implemented agent based at least partly on an emotional state of an individual. The system 400 also includes an electronic device 404 that may obtain sensor data that may be utilized to determine an emotional state of an individual 406. The individual 406 may operate the electronic device 404 to interact with a computer-implemented agent. The electronic device 404 may include a laptop computing device, a tablet computing device, a mobile communications device (e.g., a mobile phone), a wearable computing device (e.g., watch, glasses, fitness tracking device, a head mounted display, jewelry), a portable gaming device, combinations thereof, and the like.

The computing device 402 may be associated with an entity that is a service provider that provides services related to communicating information using computer-implemented agents. Additionally, the computing device 402 may be associated with a manufacturer of the electronic device 404, a distributor of the electronic device 404, or both. The computing device 402 may include one or network interfaces (not shown) to communicate with other computing devices, such as the electronic device 404, via one or more networks 408. The one or more networks 408 may include one or more of the Internet, a cable network, a satellite network, a wide area wireless communication network, a wired local area network, a wireless local area network, or a public switched telephone network (PSTN).

The computing device 402 may include one or more processors, such as processor 410. The one or more processors 410 may include at least one hardware processor, such as a microprocessor. In some cases, the one or more processors 410 may include a central processing unit (CPU), a graphics processing unit (GPU), or both a CPU and GPU, or other processing units. Additionally, the one or more processors 410 may include a local memory that may store program modules, program data, and/or one or more operating systems.

In addition, the computing device 402 may include one or more computer-readable storage media, such as computer-readable storage media 412. The computer-readable storage media 412 may include volatile and nonvolatile memory and/or removable and non-removable media implemented in any type of technology for storage of information, such as computer-readable instructions, data structures, program modules, or other data. Such computer-readable storage media 412 may include, but is not limited to, RAM, ROM, EEPROM, flash memory or other memory technology, CD-ROM, digital versatile disks (DVD) or other optical storage, magnetic cassettes, magnetic tape, solid state storage, magnetic disk storage, RAID storage systems, storage arrays, network attached storage, storage area networks, cloud storage, removable storage media, or any other medium that may be used to store the desired information and that may be accessed by a computing device. Depending on the configuration of the computing device 402, the computer-readable storage media 412 may be a type of tangible computer-readable storage media and may be a non-transitory storage media.

The computer-readable storage media 412 may be used to store any number of functional components that are executable by the one or more processors 410. In many implementations, these functional components comprise instructions or programs that are executable by the one or more processors 410 and that, when executed, implement operational logic for performing the operations attributed to the computing device 402. Functional components of the computing device 402 that may be executed on the one or more processors 410 for implementing the various functions and features related to communicating information via a computer-implemented agent based at least partly on emotional states of individuals, as described herein, include a sensor data module 414, an emotional state module 416, a communication framework module 418, an agent module 420, and a feedback module 422. One or more of the modules 414, 416, 418, 420, 422 may be used to implement the information communication system 118 of FIG. 1.

The computing device 402 may also include, or is coupled to, a data store 424 that may include, but is not limited to, RAM, ROM, EEPROM, flash memory, one or more hard disks, solid state drives, optical memory (e.g. CD, DVD), or other non-transient memory technologies. The data store 424 may maintain information that is utilized by the computing device 402 to perform operations related to communicating information via a computer-implemented agent based at least partly on emotional states of individuals. For example, the data store 424 may store emotional state benchmark data 426. In addition, the data store 424 may store communication frameworks 428.

The emotional state benchmark data 426 may include data utilized to determine an emotional state of an individual. In an example, the emotional state benchmark data 426 may include examples of sensor data that correspond to various emotional states. To illustrate, the emotional state benchmark data 426 may include images of facial features corresponding to different emotional states. In an illustrative example, the emotional state benchmark data 426 may include images of eyes, images of mouths, images of faces, images of noses, combinations thereof, and so forth that correspond to emotional states. The emotional state benchmark data 426 may also include audible data of sounds, words, or both that correspond with different emotional states. Further, the emotional state benchmark data 426 may include physiological data corresponding to one or more emotional states. In an illustrative example, the emotional state benchmark data 426 may include EEG patterns that correspond with respective emotional states. In some cases, the emotional states may be associated with identifiers.

In particular implementations, the emotional state benchmark data 426 may be organized accord to different emotional states. For example, a first portion of the emotional state benchmark data 426 may correspond to a first emotional state. To illustrate, the first portion of the emotional state benchmark data 426 may include visual data, audible data, physiological data, or combinations thereof, that correspond to the first emotional state. In another example, a second portion of the emotional state benchmark data 426 may correspond to a second emotional state. In an additional illustration, the second portion of the emotional state benchmark data 426 may include visual data, audible data, physiological data, or combinations thereof, that correspond to the second emotional state.

In some implementations, the identifiers may include at least one of “happy,” “sad,” “angry,” “surprised,” “afraid,” and the like. The emotional state benchmark data 426 may be collected by a service provider associated with the computing device 402. In other scenarios, the emotional state benchmark data 426 may be obtained from another entity, such as a research organization that gathers data (e.g., visual data, audible data, physiological data) and correlates the data with emotional states of individuals.

The communication frameworks 428 may include information related to a manner in which a computer-implemented agent communicates with individuals. In an illustrative example, the communication frameworks 428 may include the communication framework 128 of FIG. 1 and FIG. 3, the first communication framework 202 of FIG. 2, the second communication framework 206 of FIG. 2, and the modified communication framework 330 of FIG. 3. The communication frameworks 428 may each include components that determine visual characteristics of a computer-implemented agent, audible characteristics of sounds produced by a computer-implemented agent, body language of a computer-implemented agent, positioning of a computer-implemented agent, or combinations thereof.

In some implementations, one or more frameworks for communicating information may correspond to a particular emotional state. For example, one or more first communication frameworks 428 may correspond to a first emotional state and one or more second communication frameworks 428 may correspond to a second emotional state. In particular implementations, the communication frameworks 428 associated with an emotional state may be individually customized. In an illustrative example, a service provider associated with the computing device 402, or another entity, may determine default communication frameworks 428 for one or more emotional states. As the computing device 402 obtains feedback from individuals regarding interactions with a computer-implemented agent, the data store 424 may store additional communication frameworks 428 that have been modified from the default communication frameworks 428 and customized for the individuals. In some situations, the communication frameworks 428 may also be associated with content of information to be communicated to individual. Further, the communication frameworks 428 may correspond to one or more activities being performed by an individual.

The sensor data module 414 may include computer-readable instructions that are executable by the processor 410 to obtain data from one or more sensors. In some implementations, the sensor data module 414 may obtain data collected by one or more sensors of the electronic device 404. The sensor data may include visual data, such as one or more images, of the individual 406. In particular, the sensor data may include one or more images of facial features of the individual 406. The sensor data may also include audible data produced by the individual 406. For example, the sensor data may include sounds and/or words produced by the individual 406. Additionally, the sensor data may include physiological data of the individual 406. To illustrate, the sensor data may indicate heart activity of the individual 406, brain activity of the individual 406, lung activity of the individual 406, body temperature of the individual 406, skin characteristics of the individual 406, or combinations thereof. In an illustrative example, the sensor data may include EEG data of the individual 406.

The emotional state module 416 may include computer-readable instructions that are executable by the processor 410 to determine emotional states of individuals. The emotional state module 416 may determine an emotional state of an individual based at least partly on sensor data associated with the individual. Additionally, the emotional state module 416 may determine an emotional state of an individual based at least partly on an amount of correspondence between sensor data of the individual and the emotional state benchmark data 426. For example, the emotional state module 416 may compare sensor data of an individual with one or more portions of the emotional state benchmark data 426. In situations where the sensor data includes visual data, the emotional state module 416 may compare the visual data to visual data included in the emotional state benchmark data 426. Additionally, in situations where the sensor data includes audible data, the emotional state module 416 may compare the audible data to audible data included in the emotional state benchmark data 426. Further, in situations where the sensor data includes physiological data, the emotional state module 416 may compare the physiological data to physiological data included in the emotional state benchmark data 426.

The emotional state module 416 may compare sensor data to the emotional state benchmark data 426 to determine a similarity between the sensor data and one or more portions of the emotional state benchmark data 426. For example, the emotional state module 416 may perform a pattern matching analysis, an image matching analysis, or both to determine a similarity between the sensor data and a portion of the emotional state benchmark data 426. To illustrate, the emotional state module 416 may compare contours of images of one or more facial features included in the sensor data with images of facial features included in the emotional state benchmark data 426. In another illustration, the emotional state module 416 may compare EEG data from the sensor data with EEG patterns of the emotional state benchmark data 426. In an additional illustration, the emotional state module 416 may compare a pattern of wavelengths, a pattern of frequencies, or both of sounds included in the sensor data with patterns of sounds of the emotional state benchmark data 426.

The emotional state module 416 may determine that an individual is associated with an emotional state based at least partly on determining that at least a threshold amount of sensor data of the individual corresponds to one or more portions of the emotional state benchmark data 426. In some cases, the threshold amount of similarity between the sensor data and one or more portions of the emotional state benchmark data 426 may be expressed as a tolerance. For example, the tolerance may relate to at least a threshold percentage of the sensor data that corresponds to one or more portions of the emotional state benchmark data 426. In another example, the tolerance may relate to differences between the sensor data and one or more portions of the emotional state benchmark data 426 being below a threshold amount.

In an illustrative example, the emotional state module 416 may determine a similarity between an EEG pattern of the sensor data and EEG patterns of the emotional state benchmark data 426 using pattern matching techniques. The emotional state module 416 may determine that an individual is associated with a particular emotional state at least partly based on determining that the EEG pattern of the sensor data corresponds with at least a threshold amount of an EEG pattern of the emotional state benchmark data 426 associated with the particular emotional state. In another illustrative example, the emotional state module 416 may determine that an individual is associated with a particular emotional state at least partly based on determining that facial features of an individual included in one or more images correspond with at least a threshold amount of facial features included in a pattern of facial features of the emotional state benchmark data 426 associated with the particular emotional state. In a further illustrative example, the emotional state module 416 may determine that an individual is associated with a particular emotional state based at least partly on determining that a pattern of frequencies, a pattern of wavelengths, or both corresponding to sounds produced by the individual correspond with at least one of a pattern of frequencies or a pattern of wavelengths included in the emotional state benchmark data 426.

The communication framework module 418 may include computer-readable instructions that are executable by the processor 410 to determine a communication framework 428 that corresponds with an emotional state of an individual. In particular implementations, each communication framework 428 may be stored in association with one or more emotional states. For example, an identifier of one or more emotional states may be associated with each communication framework 428. The communication framework module 418 may obtain an emotional state of an individual from the emotional state module 416 and identify one or more of the communication frameworks 428 that corresponds with the emotional state based at least partly on the identifier of the emotional state. In some cases, the communication framework module 418 may also determine one or more communication frameworks 428 associated with a particular individual. To illustrate, the communication framework module 418 may determine an identifier associated with the individual 408 and identify one or more of the communication frameworks 428 that correspond to the individual 408. In an illustrative example, after obtaining an identifier of an emotional state of the individual 408 and determining an identifier of the individual 408, the framework module 418 may parse the communication frameworks 428 to identify one or more of the communication frameworks 428 that correspond with the identifier of the emotional state and the identifier of the individual 408.

In particular implementations, the communication framework module 418 may also determine a communication framework 428 based at least partly on content of information to be provided to an individual via a computer-implemented agent. For example, content of information may indicate that the information is urgent or that the content of information is positive news for the individual. The communication framework module 418 may identify a communication framework 428 that corresponds with the content of the information to be communicated to the individual. In some cases, the communication framework module 418 may utilize the emotional state of an individual and the content of information to be communicated to the individual to determine a communication framework 428 for interacting with the individual. In various implementations, the emotional state of an individual and the content of information to be communicated to the individual may each be associated with a weighting that indicates a relative importance of the emotional state of the individual and the content of information to be communicated in identifying a communication framework 428.

In addition, the communication framework module 418 may determine a communication framework 428 based at least partly on an activity being performed by an individual. For example, the communication framework module 418 may analyze sensor data indicating audible data, visual data, and/or physiological data associated with an individual and determine that the individual is participating in a particular activity. The communication framework module 418 may then identify a communication framework 428 that is associated with the individual and with the particular activity. In another example, the communication framework module 418 may analyze applications being utilized by an individual to determine an activity of the individual. To illustrate, the communication framework module 418 may obtain information from the electronic device 404 indicating that the individual 408 is utilized a particular application of the electronic device 404, such as a media player application, a navigation application, and the like. Based at least partly on the application being utilized by the individual 408, the communication framework module 418 may identify a communication framework 428 that corresponds to the individual 408 and the application being utilized by the individual 408.

The agent module 420 may include computer-readable instructions that are executable by the processor 410 to generate a representation of a computer-implemented agent. The representation of the computer-implemented agent may relate to a visible appearance of the computer-implemented agent, a location of the computer-implemented agent within an environment, body language of the computer-implemented agent, body movement of the computer-implemented agent, or combinations thereof. In addition to visible features of the computer-implemented agent, the representation of the computer-implemented agent may include or be associated with audible content produced by the computer-implemented agent. For example, the agent module 420 may determine sounds, words, voice features, or combinations thereof, that are produced in association with a computer-implemented agent.

The agent module 420 may utilize a communication framework 428 that is associated with an emotional state of an individual to generate the representation of the computer-implemented agent. For example, the agent module 420 may determine features associated with a communication framework 428 and values corresponding to each of the features of the communication framework 428. The agent module 420 may utilize the values of each of the features of the communication framework 428 to generate a representation of a computer-implemented agent. To illustrate, a communication framework 428 associated with an emotional state of the individual 408 may include voice features of tone, pitch, volume, and pace. Each of the features may be associated with a value that corresponds to a physical implementation of the feature. In an illustrative scenario, a value for the voice feature of volume may correspond to a measure of loudness of a voice of a computer-implemented agent. In another illustrative example, a value for a facial feature of eye lids may correspond to an amount that pupils that are visible. After determining the values of features of the communication framework 428 that corresponds to an emotional state of an individual, the agent module 420 may generate a representation of a computer-implemented agent that corresponds with the values of the features. In some cases, the agent module 420 may provide data corresponding to the representation to another computer device, such as the electronic device 404.

The feedback module 422 may include computer-readable instructions that are executable by the processor 410 to obtain feedback regarding interactions between a computer-implemented agent and an individual. In some cases, the feedback may include negative feedback that indicates an individual is dissatisfied with an interaction between the individual and a computer-implemented agent. In other cases, the feedback may include negative feedback that indicates an individual is satisfied with an interaction between the individual and a computer-implemented agent. The feedback module 422 may determine that the feedback includes positive feedback or negative feedback based at least partly on visual information obtained about the individual during a period of time that the feedback was received, audible information obtained about the individual during a period of time that the feedback was received, physiological information obtained about the individual during a period of time that the feedback was received, or combinations thereof. In particular implementations, the feedback module 422 may compare at least one of the visual information, the audible information, or the physiological information regarding an individual during a time that feedback is provided with previously obtained data that corresponds with positive feedback and negative feedback. In situations that the feedback module 422 determines that at least a threshold amount of the information related to the feedback corresponds with previously obtained data associated with positive feedback, the feedback module 422 may identify the feedback obtained from the individual as positive feedback. In scenarios that the feedback module 422 determines that at least a threshold amount of the information related to the feedback corresponds with previously obtained data associated with negative feedback, the feedback module 422 may identify the feedback obtained from the individual as negative feedback.

The feedback module 422 may also modify, or cause the communication framework module 418 to modify, a communication framework 428 based at least partly on the feedback obtained from an individual. In some implementations, the feedback module 422 may modify values of one or more features of a communication framework based at least partly on the feedback received from an individual. For example, the feedback module 422 may determine that a volume of the voice utilized by a computer-implemented agent was too loud or not loud enough based at least partly on the feedback received from the individual. The feedback module 422 may also determine an emotional state of the individual during a period of time that the feedback was received. Continuing with this example, the feedback module 422 may modify a communication framework 428 associated with the individual and also associated with the emotional state based on the feedback received from the individual. In a scenario where the feedback of the individual indicates that the volume of the voice utilized by a computer-implemented agent was too loud and that the emotional state of the individual was identified as “sad,” the feedback module 422 may modify a communication framework 428 associated with the emotional state of “sad” that is associated with the individual such that the voice feature of volume is reduced when the individual is determined by the emotional state module 416 to be in the emotional state of “sad.”

In various implementations, the emotional state module 416 may monitor an emotional state of an individual and determine when an emotional state of the individual changes. In instances that the emotional state module 416 determines that the emotional state of an individual has changed, the emotional state module 416 may operate in conjunction with the communication framework module 418 to determine a new communication framework 428 for the agent module 420 to utilize to generate a representation of the computer-implemented agent. In this way, as the computing device 402 may track the emotional state of an individual at various times and modify the interactions that the computer-implemented agent has with the individual based at least partly on a current emotional state of the individual.

The electronic device 404 of the system 400 may include a processor 430 and computer-readable storage media 432. The processor 430 may include a hardware-processing unit, such as a central processing unit, a graphics processing unit, or both. In an implementation, the computer-readable storage media 432 may include volatile and nonvolatile memory and/or removable and non-removable media implemented in any type of technology for storage of information, such as computer-readable instructions, data structures, program modules, or other data. Such computer-readable storage media 432 may include, but is not limited to, RAM, ROM, EEPROM, flash memory or other memory technology, CD-ROM, digital versatile disks (DVD) or other optical storage, solid state storage, magnetic disk storage, removable storage media, or any other medium that may be used to store the desired information and that can be accessed by the electronic device 404. Depending on the configuration of the electronic device 404, the computer-readable storage media 432 may be a type of tangible computer-readable storage media and may be a non-transitory storage media. The electronic device 404 may also include one or network interfaces (not shown) to communicate with other computing devices via the one or more networks 408.

The electronic device 404 may also include one or more input/output devices 434. The input/output devices 434 may include one or more sensors. In at least one example, the input/output devices 434 may include sensor(s) that may include any device or combination of devices configured to sense conditions of the individual 408 or surroundings of the individual 408. The input/output devices 434 may include one or more user facing cameras or other sensors for tracking eye movement or gaze, facial expressions, pupil dilation and/or contraction, gestures, and/or other characteristics of the user. In some examples, the input/output devices 434 may include one or more outwardly facing or environmental cameras for capturing images of real-world objects and surroundings of the individual 408. The input/output devices 434 may additionally or alternatively include one or more biometric sensors (e.g., a galvanic skin response sensor for measuring galvanic skin response, a heart rate monitor, a skin temperature sensor for measuring the temperature on the surface of the skin, an electroencephalography (EEG) device for measuring electrical activity of the brain, an electrocardiography (ECG or EKG) device for measuring electrical activity of the heart), one or more other cameras (e.g., web cameras, infrared cameras, depth cameras, etc.), microphones or other sound sensors for measuring a volume of speech, a rate of speech, etc., light sensors, optical scanners, or the like.

Individual input/output devices 434 may output data to one or more module(s) for suitable processing, such as a sensor data collection module 436, an agent representation module 438, and an individual feedback module 440. In additional and/or alternative examples, the input/output devices 434 may include any device or combination of devices configured to detect a position or movement of the electronic device 404 and other objects. For instance, the input/output devices 434 may additionally and/or alternatively include a depth map sensor, a light field sensor, a gyroscope, a sonar sensor, an infrared sensor, a compass, an accelerometer, a global positioning system (GPS) sensor, and/or any other device or component for detecting a position or movement of the electronic device 404 and/or other objects. The input/output devices 434 may also enable the generation of data characterizing interactions, such as user gestures, with the electronic device 404. For illustrative purposes, the input/output devices 434 may enable the generation of data defining a position and aspects of movement, e.g., speed, direction, acceleration, of one or more objects, which may include the electronic device 404, physical items near the electronic device 404, and/or users.

In some implementations, at least some of the input/output devices 434 may be part of, or built into, the electronic device 404. More specifically, the electronic device 404 may include a user facing camera sensor and/or an environmental camera disposed in or integrated with a nose-bridge component of the electronic device 404. As described above, the electronic device 404 may include any configuration of one or more input/output devices 434 that may be part of, or built into, the electronic device 404. However, in some examples, one or more of the input/output devices 434 may be removably coupled to the electronic device 404 or be separate from and communicatively coupled to the electronic device 404. In the latter case, data from the input/output devices 434 may be communicated from the input/output devices 434 to the electronic device 404, for example, via a wired and/or wireless network, such as network 406.

Additionally, input/output devices 434 may include one or more input interfaces that may include a keyboard, keypad, mouse, microphone, touch sensor, touch screen, joystick, control buttons, scrolling buttons, cameras, neural interface, or any other device suitable to generate a signal and/or data defining a user interaction with the electronic device 404. By way of example and not limitation, the input/output devices 434 may include a display (e.g., holographic display, head-up display, protector, touch screen, liquid crystal display (LCD), etc.), speakers, haptic interfaces, or the like.

In at least one example, a display device of the electronic device 434 may include a hardware display surface that may be configured to allow for a real-world view of an object through the hardware display surface while also providing a rendered display of computer generated content or scenes. The hardware display surface may include one or more components, such as a projector, screen, or other suitable components for producing a display of an object and/or data. In some configurations, the hardware display surface may be configured to cover at least one eye of a user. In one illustrative example, the hardware display surface may include a screen configured to cover both eyes of a user. The hardware display surface may render or cause the display of one or more images for generating a view or a stereoscopic image of one or more computer generated virtual objects. For illustrative purposes, an object can be an item, data, device, person, place, or any type of entity. In at least one example, an object can be associated with a function or a feature associated with an application. Some configurations may enable the electronic device 404 to graphically associate holographic user interfaces and other graphical elements with an object seen through a hardware display surface or rendered objects displayed on the hardware display surface of the electronic device 404.

A hardware display surface of the electronic device 404 may be configured to allow the individual 408 to view objects from different environments. In some configurations, the hardware display surface may display a rendering of a computer generated virtual object. In addition, some configurations of the hardware display surface may allow the individual 408 to see through selectable sections of the hardware display surface having a controllable level of transparency, enabling the individual 408 to view objects in his or her surrounding environment. For illustrative purposes, a perspective of the individual 408 looking at objects through the hardware display surface may be referred to herein as a “real-world view” of an object or a “real-world view of a physical object.” Computer generated renderings of objects and/or data may be displayed in, around, or near the selected portions of the hardware display surface enabling the individual 408 to view the computer generated renderings along with real-world views of objects observed through the selected portions of the hardware display surface.

Some configurations described herein provide both a “see through display” and an “augmented reality display.” For illustrative purposes, the “see through display” may include a transparent lens that may have content displayed on it. The “augmented reality display” may include an opaque display that is configured to display content over a rendering of an image, which may be from any source, such as a video feed from a camera used to capture images of an environment. For illustrative purposes, some examples described herein describe a display of rendered content over a display of an image. In addition, some examples described herein describe techniques that display rendered content over a “see through display” enabling a user to see a real-world view of an object with the content. It can be appreciated that the examples of the techniques described herein can apply to a “see through display,” an “augmented reality display,” or variations and combinations thereof. For illustrative purposes, devices configured to enable a “see through display,” “augmented reality display,” or combinations thereof are referred to herein as devices that are capable of providing a “mixed environment” or “mixed reality scene.”

As explained previously, the computer-readable storage media 432 may store a sensor data collection module 436 that is executable by the processor 430 to collect data from one or more sensors of the electronic device 404. For example, the sensor data collection module 436 may obtain visual data, such as images of the individual 408 using one or more cameras of the electronic device 408. The sensor data collection module 436 may also obtain audible data, such as sounds, words, or both from the individual 408 using one or more microphones of the electronic device 408. Additionally, the sensor data collection module 436 may obtain physiological data, such as EEG data of the individual 408. In some implementations, the sensor data collection module 436 may send data obtained from one or more sensors of the electronic device 404 to the computing device 402.

The agent representation module 438 may include computer-readable instructions that are executable by the processor 430 to generate a representation of a computer-implemented agent. The representation of the computer-implemented agent may include a physical appearance of the computer-implemented agent, movement of the computer-implemented agent, audible expressions of the computer-implemented agent, or combinations thereof. The agent representation module 438 may generate a representation of the computer-implemented agent that is visible to the individual 408 via one or more display devices of the electronic device 404. In some implementations, the agent representation module 438 may project images of a visible representation of the computer-implemented agent into an environment. In particular implementations, the agent representation module 438 may cause another computing device to produce one or more images of a computer-implemented agent. The agent representation module 438 may also produce or cause another computing device to produce one or more sounds, one or more words, or combinations thereof, with respect to the computer-implemented agent. In various implementations, the agent representation module 438 may obtain at least a portion of the data utilized to generate images of the representation of the computer-implemented agent from the computing device 402.

The agent representation module 438 may provide information utilizing the representation of a computer-implemented agent. In some cases, the computer-implemented agent may include an application executed by the electronic device 404, an application executed by the computing device 402, or both. The information communicated using the representation of the computer-implemented agent may be obtained from one or more additional applications executed by the electronic device 404. For example, the information communicated using the representation of the computer-implemented agent may be obtained from a navigational application, a search engine application, a browsing application, a social media application, combinations thereof, and the like. In other implementations, the information communicated using the representation of the computer-implemented agent may be obtained from computing devices that are remotely located from the electronic device 404. In particular implementations, the computing device 402 may provide information to the electronic device 404 that is to be communicated via a representation of a computer-implemented agent.

The individual feedback module 440 may include computer-readable instructions that are executable by the processor 430 to obtain feedback from the individual 408 regarding interactions between a computer-implemented agent and the individual 408. In some instances, the individual feedback module 430 may analyze sensor data obtained from one or more sensors of the electronic device 404 to determine that input received from the individual 408 corresponds to feedback regarding one or more interactions between the computer-implemented agent and the individual 408. In particular implementations, the individual feedback module 440 may send data obtained from one or more sensors of the electronic device 404 that correspond to feedback received from the individual 408 regarding interactions between a computer-implemented agent and the individual 408 to the computing device 402.

Although the illustrative example of FIG. 4 describes the operations of the sensor data module 414, the emotional state module 416, the communication framework module 418, the agent module 420, and the feedback module 422 as being performed by the electronic device 404, in some implementations, at least a portion of the operations performed by the modules 414, 416, 418, 420, 422 may be performed by the electronic device 404. For example, the electronic device 404 may utilize sensor data obtained via one or more sensors of the electronic device 404 to determine an emotional state of the individual 408. Additionally, the electronic device 404 may utilize an emotional state of the individual 408 to identify a communication framework associated with the emotional state and with the individual. Further, the electronic device 404 may generate a representation of a computer-implemented agent based at least partly on a communication framework. The electronic device 404 may also modify communication frameworks based at least partly on feedback received from the individual 408 regarding interactions between the individual 408 and the computer-implemented agent.

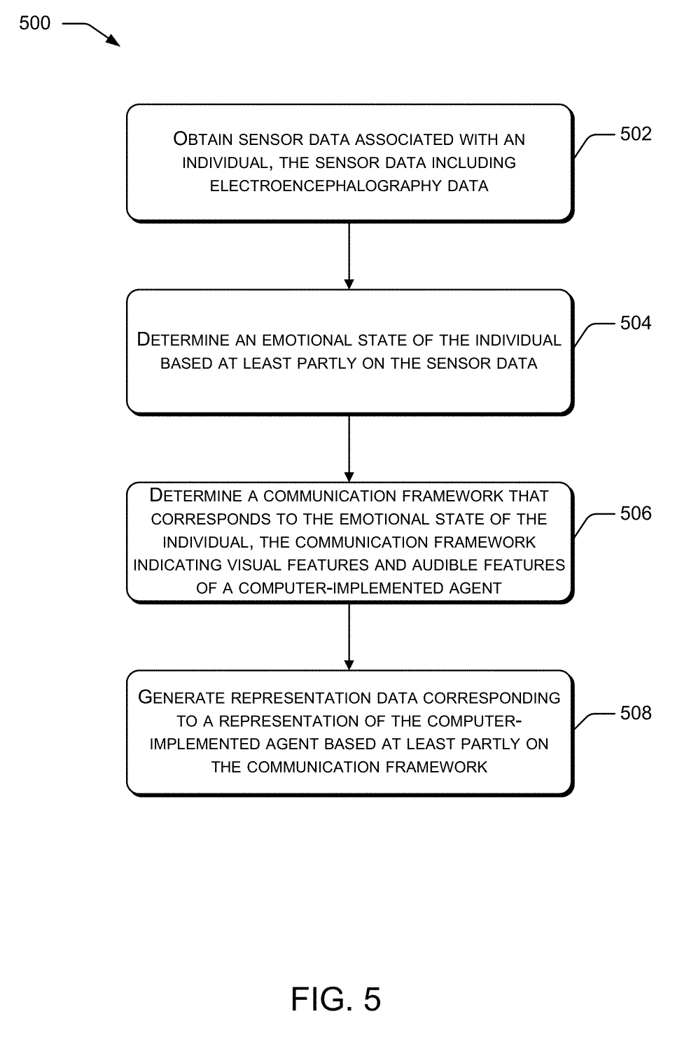

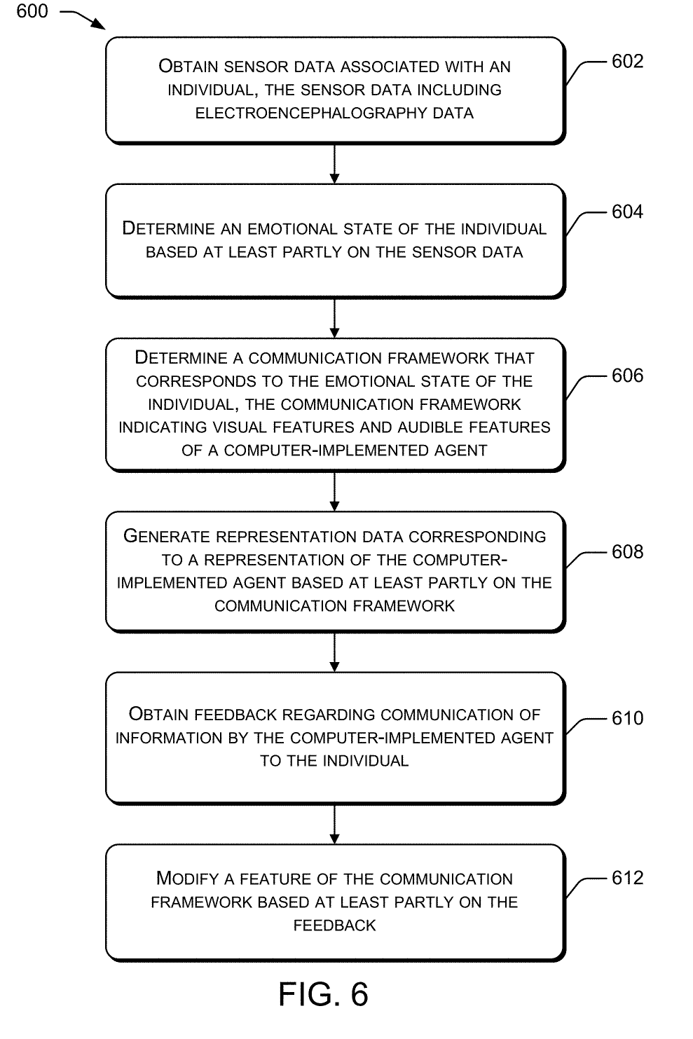

In the flow diagrams of FIGS. 5 and 6, each block represents one or more operations that may be implemented in hardware, software, or a combination thereof. In the context of software, the blocks represent computer-executable instructions that, when executed by one or more processors, cause the processors to perform the recited operations. Generally, computer-executable instructions include routines, programs, objects, modules, components, data structures, and the like that perform particular functions or implement particular abstract data types. The order in which the blocks are described is not intended to be construed as a limitation, and any number of the described operations may be combined in any order and/or in parallel to implement the processes. For discussion purposes, the processes 500 and 600 may be described with reference to FIG. 1, 2, 3 or 4 as described above, although other models, frameworks, systems and environments may implement these processes.

FIG. 5 is a flowchart of a first example process 500 to communicate information via a computer-implemented agent. At 502, the process 500 includes obtaining sensor data associated with an individual. The sensor data may include visual data, audible data, physiological data, or combinations thereof. In some cases, the physiological data may include EEG data. The sensor data may be obtained from a remotely located computing device, such as a head-mounted display computing device.

At 504, the process 500 includes determining an emotional state of the individual based at least partly on the sensor data. In some cases, determining the emotional state of the individual may include comparing the EEG data of the individual with predetermined benchmark EEG data that indicates a plurality of emotional states and determining that a threshold amount of the EEG data corresponds with a portion of the predetermined benchmark EEG data associated with the emotional state. Additionally, the sensor data may include one or more images of the individual and characteristics of one or more facial features of the individual may be determined based on the one or more images of the individual. In these situations, determining the emotional state of the individual may include comparing the characteristics of one or more facial features of the individual to predetermined benchmark image data that indicates a plurality of emotional states and determining that a threshold amount of the characteristics of the one or more facial features of the individual correspond to a portion of the predetermined benchmark image data associated with the emotional state. Furthermore, the sensor data may include audible data comprising at least one of one or more sounds or one or more words. In these scenarios, determining the emotional state of the individual based at least partly on the sensor data may include determining characteristics of one or more voice features of the individual based at least partly on the audible data and comparing the characteristics of one or more voice features of the individual to predetermined benchmark audible data that indicates a plurality of emotional states. The emotional state of the individual may be determined in response to determining that a threshold amount of the characteristics of the one or more voice features of the individual correspond to a portion of the predetermined benchmark audible data associated with the emotional state.

At 506, the process 500 includes determining a communication framework that corresponds to the emotional state of the individual. The communication framework may indicate visual features and audible features of a computer-implemented agent. In various implementations, a plurality of communication frameworks may be stored in a data store in association with the individual and the communication framework may be selected from among the plurality of communication frameworks. In some implementations, the plurality of communication frameworks may be stored in association with the individual by associating an identifier of the individual with each of the plurality of communication frameworks.

At 508, the process 500 includes generating representation data corresponding to a representation of the computer-implemented agent based at least partly on the communication framework. In some cases, the representation data may be sent to an electronic device via one or more networks. The electronic device may have provided the sensor data to a computing device performing the operations of the process 500.

In some cases, the representation data may include data corresponding to one or more images based at least partly on visible characteristics of the communication framework. The visible characteristics of the communication framework may include facial expressions, gestures, body movements, body characteristics, or combinations thereof. Additionally, the representation of the computer-implemented agent may be based at least partly on one or more sounds, one or more words, or both associated with the audible features of the communication framework. In some cases, images of the representation of the computer-implemented agent may be produced in association with one or more words or one or more sounds that indicate content of information that is to be communicated to the individual. In an illustrative example, the images of the representation of the computer-implemented agent may include one or more 3-dimensional images. In particular implementations, the information to be communicated to the individual is produced by an application executed by an electronic device in communication with the computing device via one or more networks.

In an illustrative example, a communication framework may include first values for facial features of the computer-implemented agent, second values for voice features of the computer-implemented agent, third values for body language of the computer-implemented agent, fourth values for position of the computer-implemented agent in an environment, or combinations thereof. Continuing with this example, generating a representation of a computer-implemented agent may include determining an appearance of a face of the representation of the computer-implemented agent according to the first values for the facial features of the computer-implemented agent and determining voice characteristics of the computer-implemented agent based at least partly on the second values for the voice features of the computer-implemented agent.

FIG. 6 is a flowchart of a second example process 600 to communicate information via a computer-implemented agent. At 602, the process 600 includes obtaining sensor data associated with an individual. The sensor data may include audible data, visual data, physiological data, or combinations thereof. In a particular implementation, the sensor data may include EEG data. At 604, the process 600 includes determining an emotional state of the individual based at least partly on the sensor data. In particular, the sensor data may be compared to predetermined sensor data related to a plurality of emotional states and the sensor data may be determined to correspond with at least a threshold amount of a particular emotional state.

At 606, the process 600 includes determining a communication framework that corresponds to the emotional state of the individual. The communication framework may indicate visual features and audible features of a computer-implemented agent. In addition, at 608, the process 600 includes generating representation data corresponding to a representation of the computer-implemented agent based at least partly on the communication framework. The representation of the computer-implemented agent may correspond to an appearance of the computer-implemented agent.

At 610, the process 600 includes obtaining feedback regarding communication of information by the computer-implemented agent to the individual. The feedback may be received from a computing device associated with the individual. Data received from the computing device may be identified as feedback by comparing the data to predetermined feedback data. The predetermined feedback data may indicate one or more voice features that correspond to user feedback, one or more facial features that correspond to the user feedback, one or more gestures that correspond to user feedback, one or more body movements that correspond to user feedback, or combinations thereof. In some cases, obtaining the feedback may include receiving audible information including at least one of words or sounds related to one or more interactions between the individual and the computer-implemented agent. In particular implementations, the feedback may be related to at least one of voice features of the computer-implemented agent; facial features of the computer-implemented agent; body language of the computer-implemented agent; or positioning of the computer-implemented agent within an environment that includes the individual. Furthermore, the feedback may be provided within a threshold period of time after an interaction between the computer-implemented agent and the individual. That is, the proximity in time with respect to actions of the individual with respect to an interaction between the computer-implemented agent and the individual may be less than a threshold amount of time to infer that the actions are feedback regarding the interaction. In cases where actions of the individual take place a period of time greater than the threshold period of time, the actions may not be considered to be feedback regarding an interaction between the computer-implemented agent and the individual, but may be attributed to another stimulus.

At 612, the process 600 includes modifying a feature of the communication framework based at least partly on the feedback. In various implementations, modifying the communication framework may include modifying values of the communication framework associated with at least one of voice features of the computer-implemented agent, facial features of the computer-implemented agent, body language of the computer-implemented agent, or positioning of the computer-implemented agent within the environment that includes the individual.

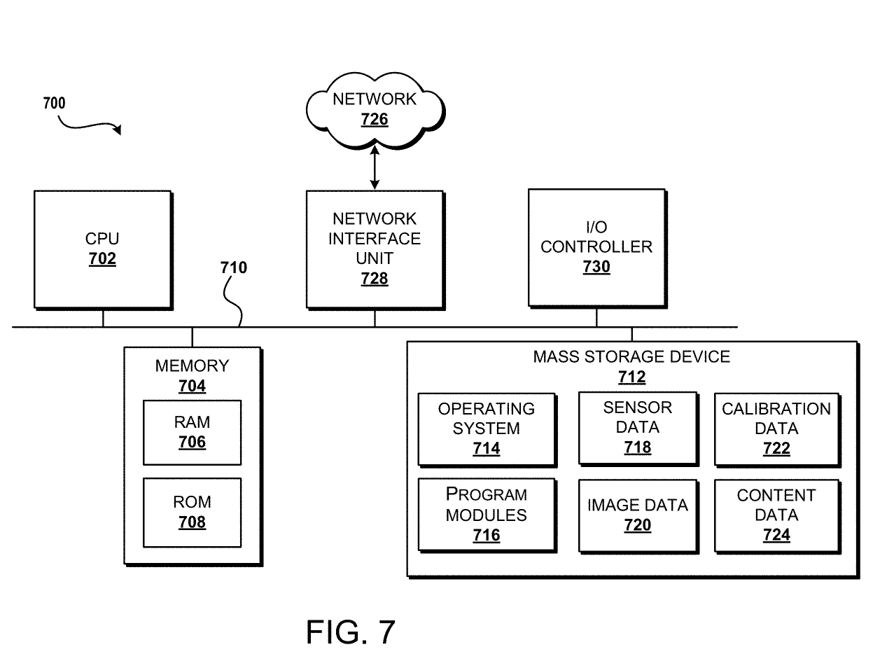

FIG. 7 shows additional details of an example computer architecture 700 for a computer, such as computing device 108, computing device 402, and/or electronic device 404, capable of executing the program components described above for utilizing computer-implemented agents to communicate information to individuals. Thus, the computer architecture 700 illustrated in FIG. 7 illustrates an architecture for a server computer, mobile phone, a PDA, a smart phone, a desktop computer, a netbook computer, a tablet computer, a laptop computer, and/or a wearable computer. The computer architecture 700 is an example architecture that may be used to execute, in whole or in part, aspects of the software components presented herein.

The computer architecture 700 illustrated in FIG. 7 includes a central processing unit 702 (“CPU”), a system memory 704, including a random access memory 706 (“RAM”) and a read-only memory (“ROM”) 708, and a system bus 710 that couples the memory 704 to the CPU 702. A basic input/output system (“BIOS”) containing the basic routines that help to transfer information between elements within the computer architecture 700, such as during startup, is stored in the ROM 708. The computer architecture 700 further includes a mass storage device 712 for storing an operating system 714, programs, module(s) 716 (e.g., the information communication system 118 of FIG. 1 and modules 414, 416, 418, 420, 422, 436, 438, and/or 440 of FIG. 4). Additionally, and/or alternatively, the mass storage device 712 may store sensor data 718, image data 720 (e.g., photographs, computer generated images, object information about real and/or virtual objects in a scene, metadata about any of the foregoing, etc.), calibration data 722, content data 724 (e.g., computer generated images, videos, scenes, etc.), and the like, as described herein.

The mass storage device 712 is connected to the CPU 702 through a mass storage controller (not shown) connected to the bus 710. The mass storage device 712 and its associated computer-readable media provide non-volatile storage for the computer architecture 700. Mass storage device 712, memory 704, computer-readable storage media 412, and computer-readable storage media 432 are examples of computer-readable media according to this disclosure. Although the description of computer-readable media contained herein refers to a mass storage device, such as a solid state drive, a hard disk or CD-ROM drive, it should be appreciated by those skilled in the art that computer-readable media may be any available computer storage media or communication media that may be accessed by the computer architecture 700.

Communication media includes computer readable instructions, data structures, program modules, or other data in a modulated data signal such as a carrier wave or other transport mechanism and includes any delivery media. The term “modulated data signal” means a signal that has one or more of its characteristics changed or set in a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of communication media.

By way of example, and not limitation, computer storage media may include volatile and non-volatile, removable and non-removable media implemented in any method or technology for storage of information such as computer-readable instructions, data structures, program modules or other data. For example, computer storage media includes, but is not limited to, RAM, ROM, erasable programmable read-only memory (“EPROM”), electrically erasable programmable read-only memory (“EEPROM”), flash memory or other solid state memory technology, compact disc read-only memory (“CD-ROM”), digital versatile disks (“DVD”), high definition/density digital versatile/video disc (“HD-DVD”), BLU-RAY disc, or other optical storage, magnetic cassettes, magnetic tape, magnetic disk storage or other magnetic storage devices, or any other medium which may be used to store the desired information and which may be accessed by the computer architecture 700. For purposes of the claims, the phrase “computer storage medium,” “computer-readable storage medium,” and variations thereof, does not include communication media.

According to various configurations, the computer architecture 700 may operate in a networked environment using logical connections to remote computers through the network 726 and/or another network (not shown). The computer architecture 700 may connect to the network 726 through a network interface unit 728 connected to the bus 710. It should be appreciated that the network interface unit 728 also may be utilized to connect to other types of networks and remote computer systems. The computer architecture 700 also may include an input/output controller 730 for receiving and processing input from input device(s) or input interface(s), and to provide output to an output device or output interface.

It should be appreciated that the software components described herein may, when loaded into the CPU 702 and executed, transform the CPU 702 and the overall computer architecture 700 from a general-purpose computing system into a special-purpose computing system customized to facilitate the functionality presented herein. The CPU 702 may be constructed from any number of transistors or other discrete circuit elements, which may individually or collectively assume any number of states. More specifically, the CPU 702 may operate as a finite-state machine, in response to executable instructions contained within the software modules described herein. These computer-executable instructions may transform the CPU 702 by specifying how the CPU 702 transitions between states, thereby transforming the transistors or other discrete hardware elements constituting the CPU 702. In some examples, processor(s) 410 and/or processor(s) 430 may correspond to CPU 702.

Encoding the software modules presented herein also may transform the physical structure of the computer-readable media presented herein. The specific transformation of physical structure may depend on various factors, in different implementations of this description. Examples of such factors may include, but are not limited to, the technology used to implement the computer-readable media, whether the computer-readable media is characterized as primary or secondary storage, and the like. For example, if the computer-readable media is implemented as semiconductor-based memory, the software described herein may be encoded on the computer-readable media by transforming the physical state of the semiconductor memory. For example, the software may transform the state of transistors, capacitors, or other discrete circuit elements constituting the semiconductor memory. The software also may transform the physical state of such components in order to store data thereupon.

As another example, the computer-readable media described herein may be implemented using magnetic or optical technology. In such implementations, the software presented herein may transform the physical state of magnetic or optical media, when the software is encoded therein. These transformations may include altering the magnetic characteristics of particular locations within given magnetic media. These transformations also may include altering the physical features or characteristics of particular locations within given optical media, to change the optical characteristics of those locations. Other transformations of physical media are possible without departing from the scope and spirit of the present description, with the foregoing examples provided only to facilitate this discussion.

In light of the above, it should be appreciated that many types of physical transformations take place in the computer architecture 700 in order to store and execute the software components presented herein. It also should be appreciated that the computer architecture 700 may include other types of computing entities, including hand-held computers, embedded computer systems, personal digital assistants, and other types of computing entities known to those skilled in the art. It is also contemplated that the computer architecture 700 may not include all of the components shown in FIG. 7, may include other components that are not explicitly shown in FIG. 7, or may utilize an architecture completely different than that shown in FIG. 7.

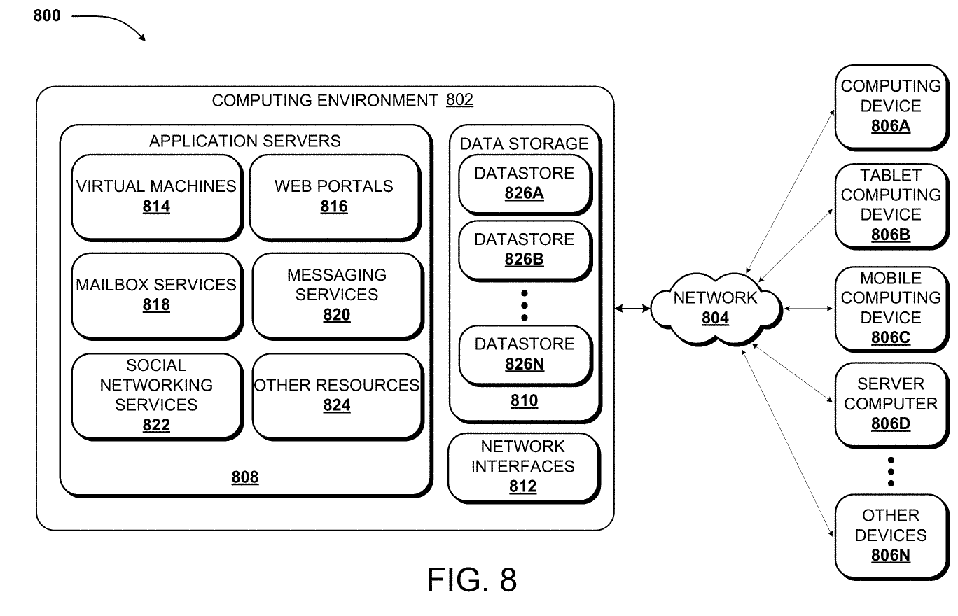

FIG. 8 depicts an example distributed computing environment 800 capable of executing the software components described herein for implementing the communication of information via computer-implemented agents. Thus, the distributed computing environment 800 illustrated in FIG. 8 may be utilized to execute any aspects of the software components presented herein to achieve aspects of the techniques described herein.

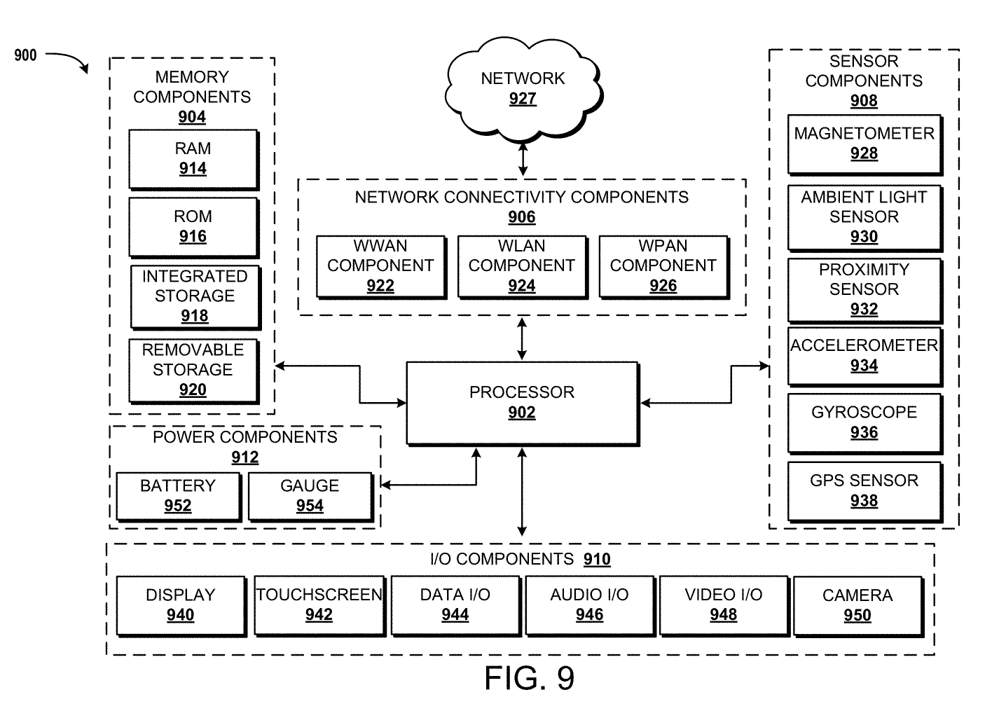

According to various implementations, the distributed computing environment 800 includes a computing environment 802 operating on, in communication with, or as part of a network 804. In at least one example, at least some of computing environment 800 may correspond to the computing device 108, computing device 402, and/or electronic device 404. The network 804 may be or may include network(s) 408 described above with reference to FIG. 4. The network 804 also may include various access networks. One or more client devices 806A-806N (hereinafter referred to collectively and/or generically as “clients 806”) may communicate with the computing environment 802 via the network 804 and/or other connections (not illustrated in FIG. 13). By way of example, computing device 108 of FIG. 1, FIG. 2, and FIG. 3 and electronic device 402 of FIG. 4 may correspond to one or more of client devices 806A-806Q (collectively referred to as “clients 806”), where Q may be any integer greater than or equal to 1 depending on the desired architecture. In one illustrated configuration, the clients 806 include a computing device 806A such as a laptop computer, a desktop computer, or other computing device, a slate or tablet computing device (“tablet computing device”) 806B, a mobile computing device 806C such as a mobile telephone, a smart phone, or other mobile computing device, a server computer 806D, a wearable computer 806E, and/or other devices 806N. It should be understood that any number of clients 806 may communicate with the computing environment 802. Two example computing architectures for the clients 806 are illustrated and described herein with reference to FIGS. 7 and 9. It should be understood that the illustrated clients 806 and computing architectures illustrated and described herein are illustrative, and should not be construed as being limited in any way.

In the illustrated configuration, the computing environment 802 includes application servers 808, data storage 810, and one or more network interfaces 812. According to various implementations, the functionality of the application servers 808 may be provided by one or more server computers that are executing as part of, or in communication with, the network 804. In some examples, the computing environment 802 may correspond to or be representative of the one or more computing devices 402 in FIG. 4, which are in communication with and accessible by the one or more electronic devices 404 via the network(s) 408 and/or 804.

In at least one example, the application servers 808 may host various services, virtual machines, portals, and/or other resources. In the illustrated configuration, the application servers 808 may host one or more virtual machines 814 for executing applications or other functionality. According to various implementations, the virtual machines 814 may execute one or more applications and/or software modules for implementing object identification using gaze tracking techniques. The application servers 808 also host or provide access to one or more portals, link pages, Web sites, and/or other information (“Web portals”) 816. The Web portals 816 may be used to communicate with one or more client computers. The application servers 808 may include one or more mailbox services 818.

According to various implementations, the application servers 808 also include one or more mailbox messaging services 820. The mailbox services 818 and/or messaging services 820 may include electronic mail (“email”) services, various personal information management (“PIM”) services (e.g., calendar services, contact management services, collaboration services, etc.), instant messaging services, chat services, forum services, and/or other communication services.

The application servers 808 also may include one or more social networking services 822. The social networking services 822 may include various social networking services including, but not limited to, services for sharing or posting status updates, instant messages, links, photos, videos, and/or other information; services for commenting or displaying interest in articles, products, blogs, or other resources; and/or other services. In some configurations, the social networking services 822 are provided by or include the FACEBOOK® social networking service, the LINKEDIN® professional networking service, the MYSPACE® social networking service, the FOURSQUARE® geographic networking service, the YAMMER® office colleague networking service, and the like. In other configurations, the social networking services 822 are provided by other services, sites, and/or providers that may or may not be explicitly known as social networking providers. For example, some web sites allow users to interact with one another via email, chat services, and/or other means during various activities and/or contexts such as reading published articles, commenting on goods or services, publishing, collaboration, gaming, and the like. Examples of such services include, but are not limited to, the WINDOWS LIVE® service and the XBOX LIVE® service from Microsoft Corporation in Redmond, Wash. Other services are possible and are contemplated.

The social networking services 822 also may include commenting, blogging, and/or micro blogging services. Examples of such services include, but are not limited to, the YELP® commenting service, the KUDZU® review service, the OFFICETALK® enterprise micro blogging service, the TWITTER® messaging service, the GOOGLE BUZZ® service, and/or other services. It should be appreciated that the above lists of services are not exhaustive and that numerous additional and/or alternative social networking services 822 are not mentioned herein for the sake of brevity. As such, the above configurations are illustrative, and should not be construed as being limited in any way. According to various implementations, the social networking services 822 may host one or more applications and/or software modules for providing the functionality described herein for providing contextually-aware location sharing services for computing devices. For instance, any one of the application servers 808 may communicate or facilitate the functionality and features described herein. For instance, a social networking application, mail client, messaging client, a browser running on a phone or any other client 1806 may communicate with a social networking service 822.

As shown in FIG. 8, the application servers 808 also may host other services, applications, portals, and/or other resources (“other resources”) 824. The other resources 824 may deploy a service-oriented architecture or any other client-server management software. It thus may be appreciated that the computing environment 802 may provide integration of the computer-implemented agent concepts and technologies described herein with various mailbox, messaging, social networking, and/or other services or resources.

As mentioned above, the computing environment 802 may include the data storage 810. According to various implementations, the functionality of the data storage 810 is provided by one or more databases operating on, or in communication with, the network 804. The functionality of the data storage 810 also may be provided by one or more server computers configured to host data for the computing environment 802. The data storage 810 may include, host, or provide one or more real or virtual containers 826A-826N (referred to collectively and/or generically as “containers 826”). Although not illustrated in FIG. 8, the containers 826 also may host or store data structures and/or algorithms for execution by one or more modules of remote computing devices (e.g., modules 414, 416, 418, 420, 422 of FIG. 4 and/or the information communication system 118 of FIG. 1). Aspects of the containers 826 may be associated with a database program, file system and/or any program that stores data with secure access features. Aspects of the containers 826 may also be implemented using products or services, such as ACTIVE DIRECTORY®, DKM®, ONEDRIVE®, DROPBOX® or GOOGLEDRIVE®.

The computing environment 802 may communicate with, or be accessed by, the network interfaces 812. The network interfaces 812 may include various types of network hardware and software for supporting communications between two or more computing entities including, but not limited to, the clients 806 and the application servers 808. It should be appreciated that the network interfaces 812 also may be utilized to connect to other types of networks and/or computer systems.

It should be understood that the distributed computing environment 800 described herein may provide any aspects of the software elements described herein with any number of virtual computing resources and/or other distributed computing functionality that may be configured to execute any aspects of the software components described herein. According to various implementations of the concepts and technologies described herein, the distributed computing environment 800 provides the software functionality described herein as a service to the clients 806. It should be understood that the clients 806 may include real or virtual machines including, but not limited to, server computers, web servers, personal computers, tablet computers, gaming consoles, smart televisions, mobile computing entities, smart phones, and/or other devices. As such, various configurations of the concepts and technologies described herein enable any device configured to access the distributed computing environment 800 to utilize the functionality described herein for providing information via a computer-implemented agent, among other aspects. In one specific example, as summarized above, techniques described herein may be implemented, at least in part, by a web browser application that may work in conjunction with the application servers 808 of FIG. 8.

FIG. 9 is an illustrative computing device architecture 900 for a computing device that is capable of executing various software components described which, in some examples, is usable to implement aspects of communicating information via a computer-implemented agent. The computing device architecture 900 is applicable to computing entities that facilitate mobile computing due, in part, to form factor, wireless connectivity, and/or battery-powered operation. In some configurations, the computing entities include, but are not limited to, mobile telephones, tablet devices, slate devices, wearable devices, portable video game devices, and the like. Moreover, aspects of the computing device architecture 900 may be applicable to traditional desktop computers, portable computers (e.g., laptops, notebooks, ultra-portables, and netbooks), server computers, and other computer systems. By way of example and not limitation, the computing device architecture 900 is applicable to any of the clients shown in FIGS. 1, 2, 3, 4, 7, and 8.

The computing device architecture 900 illustrated in FIG. 9 includes a processor 902, memory components 904, network connectivity components 906, sensor components 1408, input/output components 910, and power components 912. In the illustrated configuration, the processor 902 is in communication with the memory components 904, the network connectivity components 906, the sensor components 908, the input/output (“I/O”) components 910, and the power components 912. Although no connections are shown between the individual components illustrated in FIG. 9, the components may interact to carry out device functions. In some configurations, the components are arranged so as to communicate via one or more busses (not shown).

The processor 902 includes a central processing unit (“CPU”) configured to process data, execute computer-executable instructions of one or more application programs, and communicate with other components of the computing device architecture 900 in order to perform various functionality described herein. The processor 902 may be utilized to execute aspects of the software components presented herein. In some examples, the processor 902 may correspond to processor(s) 410, 430, and/or CPU 702, as described above in reference to FIGS. 4 and 7.

In some configurations, the processor 902 includes a graphics processing unit (“GPU”) configured to accelerate operations performed by the CPU, including, but not limited to, operations performed by executing general-purpose scientific and/or engineering computing applications, as well as graphics-intensive computing applications such as high resolution video (e.g., 1080i, 1080p, and higher resolution), video games, three-dimensional (“3D”) modeling applications, and the like. In some configurations, the processor 902 is configured to communicate with a discrete GPU (not shown). In some examples, the processor 902 may additionally or alternatively comprise a holographic processing unit (HPU) which is designed specifically to process and integrate data from multiple sensors of a head mounted computing device and to handle tasks such as spatial mapping, gesture recognition, and voice and speech recognition. In any case, the CPU, GPU, and/or HPU may be configured in accordance with a co-processing CPU/GPU/HPU computing model, wherein processing tasks are divided between the CPU, GPU, and/or HPU according to their respective strengths. For instance, the sequential part of an application may execute on the CPU, the computationally-intensive part is accelerated by the GPU, and certain specialized functions (e.g., spatial mapping, gesture recognition, and voice and speech recognition) may executed by an HPU.

In some configurations, the processor 902 is, or is included in, a System-on-Chip (“SoC”) along with one or more of the other components described herein below. For example, the SoC may include the processor 902, a GPU, one or more of the network connectivity components 906, and one or more of the sensor components 908. In some configurations, the processor 902 is fabricated, in part, utilizing a Package-on-Package (“PoP”) integrated circuit packaging technique. The processor 902 may be a single core or multi-core processor.

The processor 902 may be created in accordance with an ARM architecture, available for license from ARM HOLDINGS of Cambridge, United Kingdom. Alternatively, the processor 902 may be created in accordance with an x86 architecture, such as is available from INTEL CORPORATION of Mountain View, Calif. and others. In some configurations, the processor 902 is a SNAPDRAGON SoC, available from QUALCOMM of San Diego, Calif., a TEGRA SoC, available from NVIDIA of Santa Clara, Calif., a HUMMINGBIRD SoC, available from SAMSUNG of Seoul, South Korea, an Open Multimedia Application Platform (“OMAP”) SoC, available from TEXAS INSTRUMENTS of Dallas, Tex., a customized version of any of the above SoCs, or a proprietary SoC.

The memory components 904 include a random access memory (“RAM”) 914, a read-only memory (“ROM”) 916, an integrated storage memory (“integrated storage”) 918, and a removable storage memory (“removable storage”) 920. In some configurations, the RAM 914 or a portion thereof, the ROM 916 or a portion thereof, and/or some combination the RAM 914 and the ROM 916 is integrated in the processor 902. In some configurations, the ROM 916 is configured to store a firmware, an operating system or a portion thereof (e.g., operating system kernel), and/or a bootloader to load an operating system kernel from the integrated storage 918 and/or the removable storage 920. In some examples, memory components 904 may correspond to computer-readable media 412, computer-readable media 432, memory 704, as described above in reference to FIGS. 1, 4, and 7, respectively.

The integrated storage 918 may include a solid-state memory, a hard disk, or a combination of solid-state memory and a hard disk. The integrated storage 918 may be soldered or otherwise connected to a logic board upon which the processor 902 and other components described herein also may be connected. As such, the integrated storage 918 is integrated in the computing device. The integrated storage 918 is configured to store an operating system or portions thereof, application programs, data, and other software components described herein.

The removable storage 920 may include a solid-state memory, a hard disk, or a combination of solid-state memory and a hard disk. In some configurations, the removable storage 920 is provided in lieu of the integrated storage 918. In other configurations, the removable storage 920 is provided as additional optional storage. In some configurations, the removable storage 920 is logically combined with the integrated storage 918 such that the total available storage is made available as a total combined storage capacity. In some configurations, the total combined capacity of the integrated storage 918 and the removable storage 920 is shown to a user instead of separate storage capacities for the integrated storage 918 and the removable storage 920.

The removable storage 920 is configured to be inserted into a removable storage memory slot (not shown) or other mechanism by which the removable storage 920 is inserted and secured to facilitate a connection over which the removable storage 920 may communicate with other components of the computing device, such as the processor 902. The removable storage 920 may be embodied in various memory card formats including, but not limited to, PC card, CompactFlash card, memory stick, secure digital (“SD”), miniSD, microSD, universal integrated circuit card (“UICC”) (e.g., a subscriber identity module (“SIM”) or universal SIM (“USIM”)), a proprietary format, or the like.

It may be understood that one or more of the memory components 904 may store an operating system. According to various configurations, the operating system includes, but is not limited to, SYMBIAN OS from SYMBIAN LIMITED, WINDOWS MOBILE OS from Microsoft Corporation of Redmond, Wash., WINDOWS PHONE OS from Microsoft Corporation, WINDOWS from Microsoft Corporation, PALM WEBOS from Hewlett-Packard Company of Palo Alto, Calif., BLACKBERRY OS from Research In Motion Limited of Waterloo, Ontario, Canada, IOS from Apple Inc. of Cupertino, Calif., and ANDROID OS from Google Inc. of Mountain View, Calif. Other operating systems are also contemplated.

The network connectivity components 906 include a wireless wide area network component (“WWAN component”) 922, a wireless local area network component (“WLAN component”) 924, and a wireless personal area network component (“WPAN component”) 926. The network connectivity components 906 facilitate communications to and from the network 927 or another network, which may be a WWAN, a WLAN, or a WPAN. Although only the network 927 is illustrated, the network connectivity components 906 may facilitate simultaneous communication with multiple networks, including the network 927 of FIG. 9. For example, the network connectivity components 906 may facilitate simultaneous communications with multiple networks via one or more of a WWAN, a WLAN, or a WPAN. In some examples, the network 927 may correspond to all or part of network(s) 408, network 726, and/or network 804, as shown in FIGS. 4, 7, and 8.

The network 927 may be or may include a WWAN, such as a mobile telecommunications network utilizing one or more mobile telecommunications technologies to provide voice and/or data services to a computing device utilizing the computing device architecture 900 via the WWAN component 922. The mobile telecommunications technologies may include, but are not limited to, Global System for Mobile communications (“GSM”), Code Division Multiple Access (“CDMA”) ONE, CDMA2000, Universal Mobile Telecommunications System (“UMTS”), Long Term Evolution (“LTE”), and Worldwide Interoperability for Microwave Access (“WiMAX”). Moreover, the network 927 may utilize various channel access methods (which may or cannot be used by the aforementioned standards) including, but not limited to, Time Division Multiple Access (“TDMA”), Frequency Division Multiple Access (“FDMA”), CDMA, wideband CDMA (“W-CDMA”), Orthogonal Frequency Division Multiplexing (“OFDM”), Space Division Multiple Access (“SDMA”), and the like. Data communications may be provided using General Packet Radio Service (“GPRS”), Enhanced Data rates for Global Evolution (“EDGE”), the High-Speed Packet Access (“HSPA”) protocol family including High-Speed Downlink Packet Access (“HSDPA”), Enhanced Uplink (“EUL”) or otherwise termed High-Speed Uplink Packet Access (“HSUPA”), Evolved HSPA (“HSPA+”), LTE, and various other current and future wireless data access standards. The network 927 may be configured to provide voice and/or data communications with any combination of the above technologies. The network 927 may be configured to or adapted to provide voice and/or data communications in accordance with future generation technologies.

In some configurations, the WWAN component 922 is configured to provide dual-multi-mode connectivity to the network 927. For example, the WWAN component 922 may be configured to provide connectivity to the network 927, wherein the network 927 provides service via GSM and UMTS technologies, or via some other combination of technologies. Alternatively, multiple WWAN components 922 may be utilized to perform such functionality, and/or provide additional functionality to support other non-compatible technologies (i.e., incapable of being supported by a single WWAN component). The WWAN component 922 may facilitate similar connectivity to multiple networks (e.g., a UMTS network and an LTE network).

The network 927 may be a WLAN operating in accordance with one or more Institute of Electrical and Electronic Engineers (“IEEE”) 802.15 standards, such as IEEE 802.15a, 802.15b, 802.15g, 802.15n, and/or future 802.15 standard (referred to herein collectively as WI-FI). Draft 802.15 standards are also contemplated. In some configurations, the WLAN is implemented utilizing one or more wireless WI-FI access points. In some configurations, one or more of the wireless WI-FI access points are another computing device with connectivity to a WWAN that are functioning as a WI-FI hotspot. The WLAN component 924 is configured to connect to the network 927 via the WI-FI access points. Such connections may be secured via various encryption technologies including, but not limited, WI-FI Protected Access (“WPA”), WPA2, Wired Equivalent Privacy (“WEP”), and the like.

The network 927 may be a WPAN operating in accordance with Infrared Data Association (“IrDA”), BLUETOOTH, wireless Universal Serial Bus (“USB”), Z-Wave, ZIGBEE, or some other short-range wireless technology. In some configurations, the WPAN component 926 is configured to facilitate communications with other devices, such as peripherals, computers, or other computing entities via the WPAN.

In at least one example, the sensor components 908 may include a magnetometer 928, an ambient light sensor 930, a proximity sensor 932, an accelerometer 934, a gyroscope 936, and a Global Positioning System sensor (“GPS sensor”) 938. It is contemplated that other sensors, such as, but not limited to, temperature sensors or shock detection sensors, strain sensors, moisture sensors also may be incorporated in the computing device architecture 900.

The magnetometer 928 is configured to measure the strength and direction of a magnetic field. In some configurations the magnetometer 928 provides measurements to a compass application program stored within one of the memory components 904 in order to provide a user with accurate directions in a frame of reference including the cardinal directions, north, south, east, and west. Similar measurements may be provided to a navigation application program that includes a compass component. Other uses of measurements obtained by the magnetometer 928 are contemplated.

The ambient light sensor 930 is configured to measure ambient light. In some configurations, the ambient light sensor 930 provides measurements to an application program stored within one the memory components 904 in order to automatically adjust the brightness of a display (described below) to compensate for low-light and high-light environments. Other uses of measurements obtained by the ambient light sensor 930 are contemplated.

The proximity sensor 932 is configured to detect the presence of an object or thing in proximity to the computing device without direct contact. In some configurations, the proximity sensor 932 detects the presence of a user's body (e.g., the user's face) and provides this information to an application program stored within one of the memory components 904 that utilizes the proximity information to enable or disable some functionality of the computing device. For example, a telephone application program may automatically disable a touchscreen (described below) in response to receiving the proximity information so that the user's face does not inadvertently end a call or enable/disable other functionality within the telephone application program during the call. Other uses of proximity as detected by the proximity sensor 928 are contemplated.

The accelerometer 934 is configured to measure proper acceleration. In some configurations, output from the accelerometer 934 is used by an application program as an input mechanism to control some functionality of the application program. For example, the application program may be a video game in which a character, a portion thereof, or an object is moved or otherwise manipulated in response to input received via the accelerometer 934. In some configurations, output from the accelerometer 934 is provided to an application program for use in switching between landscape and portrait modes, calculating coordinate acceleration, or detecting a fall. Other uses of the accelerometer 934 are contemplated.

The gyroscope 936 is configured to measure and maintain orientation. In some configurations, output from the gyroscope 936 is used by an application program as an input mechanism to control some functionality of the application program. For example, the gyroscope 936 may be used for accurate recognition of movement within a 3D environment of a video game application or some other application. In some configurations, an application program utilizes output from the gyroscope 936 and the accelerometer 934 to enhance control of some functionality of the application program. Other uses of the gyroscope 936 are contemplated.

The GPS sensor 938 is configured to receive signals from GPS satellites for use in calculating a location. The location calculated by the GPS sensor 938 may be used by any application program that requires or benefits from location information. For example, the location calculated by the GPS sensor 938 may be used with a navigation application program to provide directions from the location to a destination or directions from the destination to the location. Moreover, the GPS sensor 938 may be used to provide location information to an external location-based service, such as E1515 service. The GPS sensor 938 may obtain location information generated via Wi-Fi, WIMAX, and/or cellular triangulation techniques utilizing one or more of the network connectivity components 906 to aid the GPS sensor 938 in obtaining a location fix. The GPS sensor 938 may also be used in Assisted GPS (“A-GPS”) systems.

In at least one example, the I/O components 910 may correspond to the input/output devices 434, described above with reference to FIG. 4 and/or input/output devices described with respect to FIG. 7. Additionally, and/or alternatively, the I/O components may include a display 940, a touchscreen 942, a data I/O interface component (“data I/O”) 944, an audio I/O interface component (“audio I/O”) 946, a video I/O interface component (“video I/O”) 948, and a camera 950. In some configurations, the display 940 and the touchscreen 942 are combined. In some configurations two or more of the data I/O component 944, the audio I/O component 946, and the video I/O component 948 are combined. The I/O components 910 may include discrete processors configured to support the various interface described below, or may include processing functionality built-in to the processor 902.

The display 940 is an output device configured to present information in a visual form. In particular, the display 940 may present graphical user interface (“GUI”) elements, text, images, video, notifications, virtual buttons, virtual keyboards, messaging data, Internet content, device status, time, date, calendar data, preferences, map information, location information, and any other information that is capable of being presented in a visual form. In some configurations, the display 940 is a liquid crystal display (“LCD”) utilizing any active or passive matrix technology and any backlighting technology (if used). In some configurations, the display 940 is an organic light emitting diode (“OLED”) display. In some configurations, the display 940 is a holographic display. Other display types are contemplated.

In at least one example, the display 940 may correspond to a hardware display surface of the computing device 108 and/or the electronic device 404. As described above, the hardware display surface may be configured to graphically associate holographic user interfaces and other graphical elements with an object seen through the hardware display surface or rendered objects displayed on the hardware display surface.

The touchscreen 942, also referred to herein as a “touch-enabled screen,” is an input device configured to detect the presence and location of a touch. The touchscreen 942 may be a resistive touchscreen, a capacitive touchscreen, a surface acoustic wave touchscreen, an infrared touchscreen, an optical imaging touchscreen, a dispersive signal touchscreen, an acoustic pulse recognition touchscreen, or may utilize any other touchscreen technology. In some configurations, the touchscreen 942 is incorporated on top of the display 940 as a transparent layer to enable a user to use one or more touches to interact with objects or other information presented on the display 940. In other configurations, the touchscreen 942 is a touch pad incorporated on a surface of the computing device that does not include the display 940. For example, the computing device may have a touchscreen incorporated on top of the display 940 and a touch pad on a surface opposite the display 940.

In some configurations, the touchscreen 942 is a single-touch touchscreen. In other configurations, the touchscreen 942 is a multi-touch touchscreen. In some configurations, the touchscreen 942 is configured to detect discrete touches, single touch gestures, and/or multi-touch gestures. These are collectively referred to herein as gestures for convenience. Several gestures will now be described. It should be understood that these gestures are illustrative and are not intended to limit the scope of the appended claims. Moreover, the described gestures, additional gestures, and/or alternative gestures may be implemented in software for use with the touchscreen 942. As such, a developer may create gestures that are specific to a particular application program.

In some configurations, the touchscreen 942 supports a tap gesture in which a user taps the touchscreen 942 once on an item presented on the display 940. The tap gesture may be used to perform various functions including, but not limited to, opening or launching whatever the user taps. In some configurations, the touchscreen 942 supports a double tap gesture in which a user taps the touchscreen 942 twice on an item presented on the display 940. The double tap gesture may be used to perform various functions including, but not limited to, zooming in or zooming out in stages. In some configurations, the touchscreen 942 supports a tap and hold gesture in which a user taps the touchscreen 942 and maintains contact for at least a pre-defined time. The tap and hold gesture may be used to perform various functions including, but not limited to, opening a context-specific menu.

In some configurations, the touchscreen 942 supports a pan gesture in which a user places a finger on the touchscreen 942 and maintains contact with the touchscreen 942 while moving the finger on the touchscreen 942. The pan gesture may be used to perform various functions including, but not limited to, moving through screens, images, or menus at a controlled rate. Multiple finger pan gestures are also contemplated. In some configurations, the touchscreen 942 supports a flick gesture in which a user swipes a finger in the direction the user wants the screen to move. The flick gesture may be used to perform various functions including, but not limited to, scrolling horizontally or vertically through menus or pages. In some configurations, the touchscreen 942 supports a pinch and stretch gesture in which a user makes a pinching motion with two fingers (e.g., thumb and forefinger) on the touchscreen 942 or moves the two fingers apart. The pinch and stretch gesture may be used to perform various functions including, but not limited to, zooming gradually in or out of a website, map, or picture.

Although the above gestures have been described with reference to the use of one or more fingers for performing the gestures, other appendages such as toes or objects such as styluses may be used to interact with the touchscreen 942. As such, the above gestures should be understood as being illustrative and should not be construed as being limited in any way.

The data I/O interface component 944 is configured to facilitate input of data to the computing device and output of data from the computing device. In some configurations, the data I/O interface component 944 includes a connector configured to provide wired connectivity between the computing device and a computer system, for example, for synchronization operation purposes. The connector may be a proprietary connector or a standardized connector such as USB, micro-USB, mini-USB, or the like. In some configurations, the connector is a dock connector for docking the computing device with another device such as a docking station, audio device (e.g., a digital music player), or video device.

The audio I/O interface component 946 is configured to provide audio input and/or output capabilities to the computing device. In some configurations, the audio I/O interface component 946 includes a microphone configured to collect audio signals. In some configurations, the audio I/O interface component 946 includes a headphone jack configured to provide connectivity for headphones or other external speakers. In some configurations, the audio I/O interface component 946 includes a speaker for the output of audio signals. In some configurations, the audio I/O interface component 946 includes an optical audio cable out.

The video I/O interface component 948 is configured to provide video input and/or output capabilities to the computing device. In some configurations, the video I/O interface component 948 includes a video connector configured to receive video as input from another device (e.g., a video media player such as a DVD or BLURAY player) or send video as output to another device (e.g., a monitor, a television, or some other external display). In some configurations, the video I/O interface component 948 includes a High-Definition Multimedia Interface (“HDMI”), mini-HDMI, micro-HDMI, DisplayPort, or proprietary connector to input/output video content. In some configurations, the video I/O interface component 948 or portions thereof is combined with the audio I/O interface component 946 or portions thereof.

The camera 950 may be configured to capture still images and/or video. The camera 950 may utilize a charge coupled device (“CCD”) or a complementary metal oxide semiconductor (“CMOS”) image sensor to capture images. In some configurations, the camera 950 includes a flash to aid in taking pictures in low-light environments. Settings for the camera 950 may be implemented as hardware or software buttons. Images and/or video captured by camera 950 may additionally or alternatively be used to detect non-touch gestures, facial expressions, eye movement, or other movements and/or characteristics of the user.

Although not illustrated, one or more hardware buttons may also be included in the computing device architecture 900. The hardware buttons may be used for controlling some operational aspect of the computing device. The hardware buttons may be dedicated buttons or multi-use buttons. The hardware buttons may be mechanical or sensor-based.

The illustrated power components 912 include one or more batteries 952, which may be connected to a battery gauge 954. The batteries 952 may be rechargeable or disposable. Rechargeable battery types include, but are not limited to, lithium polymer, lithium ion, nickel cadmium, and nickel metal hydride. Each of the batteries 952 may be made of one or more cells.

The battery gauge 954 may be configured to measure battery parameters such as current, voltage, and temperature. In some configurations, the battery gauge 954 is configured to measure the effect of a battery's discharge rate, temperature, age and other factors to predict remaining life within a certain percentage of error. In some configurations, the battery gauge 954 provides measurements to an application program that is configured to utilize the measurements to present useful power management data to a user. Power management data may include one or more of a percentage of battery used, a percentage of battery remaining, a battery condition, a remaining time, a remaining capacity (e.g., in watt hours), a current draw, and a voltage.

The power components 912 may also include a power connector, which may be combined with one or more of the aforementioned I/O components 910. The power components 912 may interface with an external power system or charging equipment via a power I/O component.

### Example Clauses

The disclosure presented herein can be considered in view of the following clauses.

A. A computing device comprising: one or more processors; and one or more computer-readable storage media storing instructions that are executable by the one or more processors to perform operations comprising: identifying information to communicate to an individual; obtaining electroencephalography (EEG) data of the individual, the EEG data including a pattern of EEG data over a period of time; determining, based at least partly on the EEG data, an emotional state of the individual during the period of time; identifying a plurality of communication frameworks that are stored in a data store in association with the individual, a communication framework of the plurality of communication frameworks indicating visual features and audible features of a computer-implemented agent; determining that the communication framework corresponds to the emotional state of the individual; and generating representation data indicating a representation of the computer-implemented agent based at least partly on the communication framework.

B. The computing device of clause A, wherein the representation data corresponds to one or more images of the representation of the computer-implemented agent, the one or more images including the visual features of the communication framework, and the visual features including facial expressions, gestures, body movements, body characteristics, or combinations thereof.

C. The computing device of clause A or B, wherein the representation data corresponds to one or more sounds, one or more words, or both of the representation of the computer-implemented agent, the one or more sounds, the one or more words, or both are based at least partly on the audible features of the communication framework.

D. The computing device of any one of clauses A-C, wherein generating the representation data of the computer-implemented agent includes determining one or more words according to the information to communicate to the individual.

E. The computing device of any one of clauses A-D, wherein the information to be communicated to the individual is produced by an application executed by an electronic device in communication with the computing device via one or more networks.

F. The computing device of any one of clauses A-E, wherein storing the plurality of communication frameworks in association with the individual includes associating an identifier of the individual with each of the plurality of communication frameworks.

G. The computing device of any one of clauses A-F, wherein one or more images of the representation of the computer-implemented agent include one or more 3-dimensional images.

H. The computing device of any one of clauses A-G, wherein the operations further comprise: obtaining feedback regarding one or more interactions between the individual and the computer-implemented agent related to the computer-implemented agent providing the information to be communicated to the individual; and modifying a feature of the communication framework based at least partly on the feedback.

I. The computing device of any one of clauses A-H, wherein the communication framework includes first values for facial features of the computer-implemented agent and second values for voice features of the computer-implemented agent.

J. The computing device of clause I, wherein the communication framework includes third values for body language of the computer-implemented agent and fourth values for position of the computer-implemented agent in an environment that includes the individual.

K. The computing device of clause I, wherein generating the representation data of the computer-implemented agent includes determining an appearance of a face of the representation of the computer-implemented agent according to the first values for the facial features of the computer-implemented agent and determining voice characteristics of the computer-implemented agent based at least partly on the second values for the voice features of the computer-implemented agent.

L. The computing device of any one of clauses A-K, wherein: a first communication framework of the plurality of communication frameworks corresponds to a first emotional state and is associated with a first pattern of EEG data; a second communication framework of the plurality of communication frameworks corresponds to a second emotional state and is associated with a second pattern of EEG data different from the first pattern of EEG data; and the method further comprises: comparing the EEG data of the individual to the first pattern of EEG data and the second pattern of EEG data; and determining the emotional state of the individual during the period of time includes determining that a threshold amount of the EEG data of the individual corresponds to the first pattern of EEG data.

M. A method comprising: obtaining, by a computing device including a processor and memory, sensor data for an individual, the sensor data including electroencephalography (EEG) data; determining, by the computing device, an emotional state of the individual based at least partly on the sensor data; determining, by the computing device, a communication framework that corresponds to the emotional state of the individual, the communication framework indicating visual features and audible features of a computer-implemented agent; and generating, by the computing device, representation data indicating a representation of the computer-implemented agent based at least partly on the communication framework.

N. The method of clause M, wherein determining the emotional state of the individual includes: comparing the EEG data with predetermined benchmark EEG data that indicates a plurality of emotional states; and determining that a threshold amount of the EEG data corresponds with a portion of the predetermined benchmark EEG data associated with the emotional state.

O. The method of clause M or N, wherein: the sensor data includes one or more images of the individual; and determining the emotional state of the individual based at least partly on the sensor data further comprises: determining, based at least partly on the one or more images of the individual, characteristics of one or more facial features of the individual; comparing the characteristics of one or more facial features of the individual to predetermined benchmark image data that indicates a plurality of emotional states; and determining that a threshold amount of the characteristics of the one or more facial features of the individual correspond to a portion of the predetermined benchmark image data associated with the emotional state.

P. The method of any one of clauses M-O, wherein: the sensor data includes audible data of the individual, the audible data including at least one of one or more sounds or one or more words; and determining the emotional state of the individual based at least partly on the sensor data further comprises: determining characteristics of one or more voice features of the individual based at least partly on the audible data; comparing the characteristics of the one or more voice features of the individual to predetermined benchmark audible data that indicates a plurality of emotional states; and determining that a threshold amount of the characteristics of the one or more voice features of the individual correspond to a portion of the predetermined benchmark audible data associated with the emotional state.

Q. The method of any one of clauses M-P, wherein the sensor data is obtained from an electronic device via one or more networks, and the method further comprises: sending the representation data to the electronic device.

R. The method of clause Q, further comprising: receiving feedback from the electronic device, the feedback corresponding to one or more interactions between the individual and the computer-implemented agent; and modifying the communication framework based at least partly on the feedback.

S. The method of clause R, wherein receiving feedback from the electronic device includes receiving audible information including at least one of words or sounds related to the one or more interactions between the individual and the computer-implemented agent.

T. A computing device comprising: one or more processors; and one or more computer-readable storage media storing instructions that are executable by the one or more processors to perform operations comprising: obtaining the sensor data including at least one of visual data associated with an individual, audible data associated with the individual, or electroencephalography (EEG) data associated with the individual; determining an emotional state of the individual based at least partly on the sensor data; determining a communication framework that corresponds to the emotional state of the individual, the communication framework indicating visual features and audible features of a computer-implemented agent; and generating representation data indicating a representation of the computer-implemented agent based at least partly on the communication framework; obtaining feedback regarding communication of information by the computer-implemented agent to the individual; and modifying a feature of the communication framework based at least partly on the feedback.

U. The computing device of clause T, wherein the operations further comprise: obtaining data from an electronic device, the data indicating the feedback of the individual regarding one or more interactions between the computer-implemented agent and the individual.

V. The computing device of clause U, wherein the operations further comprise: determining that the data obtained from the electronic device is associated with the feedback by comparing the data to predetermined feedback data, the predetermined feedback data indicating one or more voice features that correspond to user feedback, one or more facial features that correspond to the user feedback, one or more gestures that correspond to user feedback, one or more body movements that correspond to user feedback, or combinations thereof.

W. The computing device of any one of clauses T-V, wherein the feedback is related to at least one of: voice features of the computer-implemented agent; facial features of the computer-implemented agent; body language of the computer-implemented agent; or positioning of the computer-implemented agent within an environment that includes the individual.

X. The computing device of clause W, wherein modifying the communication framework based at least partly on the feedback includes modifying values of the communication framework associated with at least one of the voice features of the computer-implemented agent, the facial features of the computer-implemented agent, the body language of the computer-implemented agent, or the positioning of the computer-implemented agent within the environment that includes the individual.

Y. The computing device of any one of clauses T-X, wherein obtaining the feedback includes determining that the feedback is provided within a threshold period of time after an interaction between the computer-implemented agent and the individual.

Although various embodiments of the method and apparatus of the present invention have been illustrated herein in the Drawings and described in the Detailed Description, it will be understood that the invention is not limited to the embodiments disclosed, but is capable of numerous rearrangements, modifications and substitutions without departing from the scope of the present disclosure.

What is claimed is:

1. A computing device comprising:
one or more processors; and
one or more computer-readable storage media storing instructions that are executable by the one or more processors to perform operations comprising:
identifying information to communicate to an individual;
obtaining electroencephalography (EEG) data of the individual, the EEG data including a pattern of EEG data over a period of time;
determining, based at least partly on the EEG data, an emotional state of the individual during the period of time;
identifying a plurality of communication frameworks that are stored in a data store in association with the individual, a communication framework of the plurality of communication frameworks indicating visual features and audible features of a computer-implemented agent;
determining that the communication framework corresponds to the emotional state of the individual; and
generating representation data indicating a representation of the computer-implemented agent based at least partly on the communication framework.

2. The computing device of claim 1, wherein the representation data corresponds to one or more images of the representation of the computer-implemented agent, the one or more images including the visual features of the communication framework, and the visual features including facial expressions, gestures, body movements, body characteristics, or combinations thereof.

3. The computing device of claim 1, wherein the representation data corresponds to one or more sounds, one or more words, or both of the representation of the computer-implemented agent, the one or more sounds, the one or more words, or both are based at least partly on the audible features of the communication framework.

4. The computing device of claim 1, wherein the operations further comprise:
obtaining feedback regarding one or more interactions between the individual and the computer-implemented agent related to the computer-implemented agent providing the information to be communicated to the individual; and
modifying a feature of the communication framework based at least partly on the feedback.

5. The computing device of claim 1, wherein the communication framework includes first values for facial features of the computer-implemented agent and second values for voice features of the computer-implemented agent.

6. The computing device of claim 5, wherein generating the representation data of the computer-implemented agent includes determining an appearance of a face of the representation of the computer-implemented agent according to the first values for the facial features of the computer-implemented agent and determining voice characteristics of the computer-implemented agent based at least partly on the second values for the voice features of the computer-implemented agent.

7. The computing device of claim 1, wherein:
a first communication framework of the plurality of communication frameworks corresponds to a first emotional state and is associated with a first pattern of EEG data;
a second communication framework of the plurality of communication frameworks corresponds to a second emotional state and is associated with a second pattern of EEG data different from the first pattern of EEG data; and
the method further comprises:
comparing the EEG data of the individual to the first pattern of EEG data and the second pattern of EEG data; and
determining the emotional state of the individual during the period of time includes determining that a threshold amount of the EEG data of the individual corresponds to the first pattern of EEG data.

8. A method comprising:
obtaining, by a computing device including a processor and memory, sensor data for an individual, the sensor data including electroencephalography (EEG) data;
determining, by the computing device, an emotional state of the individual based at least partly on the sensor data;
determining, by the computing device, a communication framework that corresponds to the emotional state of the individual, the communication framework indicating visual features and audible features of a computer-implemented agent; and
generating, by the computing device, representation data indicating a representation of the computer-implemented agent based at least partly on the communication framework.

9. The method of claim 8, wherein determining the emotional state of the individual includes:
comparing the EEG data with predetermined benchmark EEG data that indicates a plurality of emotional states; and
determining that a threshold amount of the EEG data corresponds with a portion of the predetermined benchmark EEG data associated with the emotional state.

10. The method of claim 8, wherein:
the sensor data includes one or more images of the individual; and
determining the emotional state of the individual based at least partly on the sensor data further comprises:
determining, based at least partly on the one or more images of the individual, characteristics of one or more facial features of the individual;
comparing the characteristics of the one or more facial features of the individual to predetermined benchmark image data that indicates a plurality of emotional states; and
determining that a threshold amount of the characteristics of the one or more facial features of the individual correspond to a portion of the predetermined benchmark image data associated with the emotional state.

11. The method of claim 8, wherein:
the sensor data includes audible data of the individual, the audible data including at least one of one or more sounds or one or more words; and
determining the emotional state of the individual based at least partly on the sensor data further comprises:
determining characteristics of one or more voice features of the individual based at least partly on the audible data;
comparing the characteristics of the one or more voice features of the individual to predetermined benchmark audible data that indicates a plurality of emotional states; and
determining that a threshold amount of the characteristics of the one or more voice features of the individual correspond to a portion of the predetermined benchmark audible data associated with the emotional state.

12. The method of claim 8, wherein the sensor data is obtained from an electronic device via one or more networks, and the method further comprises:
sending the representation data to the electronic device.

13. The method of claim 12, further comprising:
receiving feedback from the electronic device, the feedback corresponding to one or more interactions between the individual and the computer-implemented agent; and
modifying the communication framework based at least partly on the feedback.

14. The method of claim 13, wherein receiving feedback from the electronic device includes receiving audible information including at least one of words or sounds related to the one or more interactions between the individual and the computer-implemented agent.

15. A computing device comprising:
one or more processors; and
one or more computer-readable storage media storing instructions that are executable by the one or more processors to perform operations comprising:
obtaining sensor data including at least one of visual data associated with an individual, audible data associated with the individual, or electroencephalography (EEG) data associated with the individual;
determining an emotional state of the individual based at least partly on the sensor data;
determining a communication framework that corresponds to the emotional state of the individual, the communication framework indicating visual features and audible features of a computer-implemented agent;
generating representation data indicating a representation of the computer-implemented agent based at least partly on the communication framework;
obtaining feedback regarding communication of information by the computer-implemented agent to the individual; and
modifying a feature of the communication framework based at least partly on the feedback.

16. The computing device of claim 15, wherein the operations further comprise:
obtaining data from an electronic device, the data indicating the feedback of the individual regarding one or more interactions between the computer-implemented agent and the individual.

17. The computing device of claim 16, wherein the operations further comprise:
determining that the data obtained from the electronic device is associated with the feedback by comparing the data to predetermined feedback data, the predetermined feedback data indicating one or more voice features that correspond to user feedback, one or more facial features that correspond to user feedback, one or more gestures that correspond to user feedback, one or more body movements that correspond to user feedback, or combinations thereof.

18. The computing device of claim 15, wherein the feedback is related to at least one of:
voice features of the computer-implemented agent;
facial features of the computer-implemented agent;
body language of the computer-implemented agent; or
positioning of the computer-implemented agent within an environment that includes the individual.

19. The computing device of claim 18, wherein modifying the feature of the communication framework based at least partly on the feedback includes modifying values of the communication framework associated with at least one of the voice features of the computer-implemented agent, the facial features of the computer-implemented agent, the body language of the computer-implemented agent, or the positioning of the computer-implemented agent within the environment that includes the individual.

20. The computing device of claim 15, wherein obtaining the feedback includes determining that the feedback is provided within a threshold period of time after an interaction between the computer-implemented agent and the individual.
